(19) United States
(12) Patent Application Publication
(10) Pub. No.: US 2004/0198758 A1
(43) Pub. Date: Oct. 7, 2004

(54) N-heterocyclyl substituted thienyloxy-pyrimidines used as herbicides

(76) Inventors: Liliana Parra Rapado, Mannheim (DE); Wolfgang von Deyn, Neustadt (DE); Michael Hofmann, Ludwigshafen (DE); Ernst Baumann, Dudenhofen (DE); Markus Kordes, Frankenthal (DE); Ulf Misslitz, Neustad (DE); Cyrill Zagar, Mannheim (DE); Matthias Witschel, Bad Durkheim (DE); Andreas Landes, Romerberg Heiligenstein (DE)

Correspondence Address: KEIL & WEINKAUF, 1350 CONNECTICUT AVENUE, N.W., WASHINGTON, DC, 20036, US

(21) Appl. No.: 10/486,398

(22) PCT Filed: Jul. 7, 2002
(86) PCT No.: PCT/EP02/08451

(30) Foreign Application Priority Data
Aug. 17, 2001 (DE) 101394047

(51) Int. Cl. A61K 31/513; C 07D 4 3/14

(52) U.S. Cl. 514/269; 544/310

(57) ABSTRACT

N-heterocyclyl-substituted thienyloxypyrimidines of the formula I

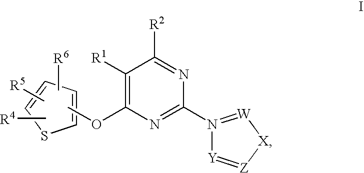

where:

W, X, Y, Z independently of one another are N or CR³, where at least one of the variables is CR³;

R¹ is hydrogen, halogen, cyano, alkyl, haloalkyl, alkoxy or haloalkoxy;

R² is hydrogen, halogen, cyano, alkyl, alkenyl, alkynyl, haloalkyl, haloalkenyl, haloalkynyl, alkoxy, alkenyloxy, alkynyloxy, haloalkoxy, alkoxyalkyl, alkylamino, dialkylamino, alkylthio, haloalkylthio, alkylsulfinyl, haloalkylsulfinyl, alkylsulfonyl, haloalkylsulfonyl, COOR⁷ or CONR⁸R⁹;

R³ is hydrogen, halogen, cyano, nitro, alkyl, haloalkyl, alkoxy, haloalkoxy, alkylthio or haloalkylthio, alkylsulfonyl or COOR⁷

R⁴, R⁵, R⁶ are hydrogen, halogen, cyano, alkyl, haloalkyl, alkoxy, haloalkoxy, alkylthio, haloalkylthio, alkylsulfonyl or haloalkylsulfonyl;

R⁷ is hydrogen, alkyl, alkenyl, alkynyl or haloalkyl;

R⁸ is hydrogen, alkyl, alkenyl, alkynyl or alkoxy;

R⁹ is hydrogen, alkyl, alkenyl or alkynyl;

and their agriculturally useful salts;

processes and intermediates for their preparation; and the use of these compounds or of compositions comprising these compounds for controlling undesirable plants are described.

The present invention relates to N-heterocyclyl-substituted thienyloxypyrimidines of the formula I

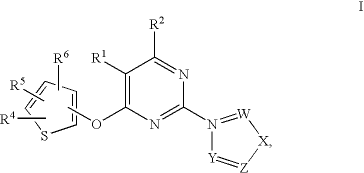

where:

W, X, Y, Z independently of one another are N or CR³, where at least one of the variables is CR³;

R¹ is hydrogen, halogen, cyano, Cₗ-C₆-alkyl, C₁-C₆-haloalkyl, C₁-C₆-alkoxy or C₁-C₆-haloalkoxy;

R² is hydrogen, halogen, cyano, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₃-C₆-alkenyloxy, C₃-C₆-alkynyloxy, Cₗ-C₆-haloalkoxy, Cₗ-C₆-alkoxy-Cₗ-C₄-alkyl, C₁-C₆-alkylamino, di-(C₁-C₄-alkyl)amino, C₁-C₆-alkylthio, C₁-C₆-haloalkylthio, C₁-C₆-alkylsulfinyl, C₁-C₆-haloalkylsulfinyl, Cₗ-C₆-alkylsulfonyl, C₁-C₆-haloalkylsulfonyl, COOR⁷ or CONR⁸R⁹;

R³ is hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or COOR⁷;

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or C₁-C₆-haloalkylsulfonyl;

R⁷ is hydrogen, C₁-C₄-alkyl, C₃-C₄-alkenyl, C₃-C₄-alkynyl or C₁-C₄-haloalkyl;

R⁸ is hydrogen, C₁-C₄-alkyl, C₃-C₄-alkenyl, C₃-C₄-alkynyl or C₁-C₄-alkoxy;

R⁹ is hydrogen, C₁-C₄-alkyl, C₃-C₄-alkenyl or C₃-C₄-alkynyl;

and their agriculturally useful salts.

Moreover, the invention relates to intermediates and processes for preparing compounds of the formula I, to compositions comprising them and to the use of these derivatives or of the compositions comprising these derivatives for controlling harmful plants.

WO 98/40379 describes heteroarylazole herbicides. WO 99/24427 discloses herbicidally active furanyl- and thienyloxyazines.

However, the herbicidal properties of the prior-art compounds and/or their compatibility with crop plants are not entirely satisfactory.

It is an object of the present invention to provide in particular herbicidally active compounds having improved properties.

We have found that this object is achieved by the N-heterocyclyl-substituted thienyloxypyrimidines of the formula I and their herbicidal action.

Furthermore, we have found herbicidal compositions which comprise the compounds I and have very good herbicidal action. Moreover, we have found processes for preparing these compositions and methods for controlling undesirable vegetation using the compounds I.

Depending on the substitution pattern, the compounds of the formula I may contain one or more centers of chirality, in which case they are present as enantiomers or mixtures of diastereomers. The invention provides both the pure enantiomers or diastereomers and their mixtures.

The compounds of the formula I can also be present in the form of their agriculturally useful salts, the type of salt generally being immaterial. Suitable are, in general, the salts of those cations and the acid addition salts of those acids whose cations and anions, respectively, do not adversely affect the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, 1 to 4 hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di-(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri (C₁-C₄-alkyl) sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and also the anions of C₁-C₄-alkanoic acids, preferably formate, acetate, propionate and butyrate.

The organic moieties mentioned for the substituents R¹-R⁹ are collective terms for individual enumerations of the individual group members. All hydrocarbon chains, i.e. all alkyl, haloalkyl, alkoxy, haloalkoxy, alkylthio, haloalkylthio, alkylsulfinyl, haloalkylsulfinyl, alkylsulfonyl, haloalkylsulfonyl, alkenyloxy, alkynyloxy, alkylamino and dialkylamino moieties can be straight-chain or branched. Unless indicated otherwise, halogenated substituents preferably carry one to five, in particular one to three, identical or different halogen atoms. The term ‘halogen’ denotes in each case fluorine, chlorine, bromine or iodine.

Examples of other meanings are:

C₁-C₄-alkyl and the alkyl moieties of hydroxy-C₁-C₄-alkyl, hydroxy-C₁-C₄-alkoxy-C₁-C₄-alkyl, tri(C₁-C₄-alkyl)sulfonium and tri(C₁-C₄-alkyl)sulfoxonium: for example methyl, ethyl, propyl, 1-methylethyl, butyl, 1-methylpropyl, 2-methylpropyl and 1,1-dimethylethyl;

C₁-C₆-alkyl: C₁-C₄-alkyl as mentioned above, and also, for example, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;

C₂-C₆-alkenyl: for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

C₂-C₆-alkynyl: for example ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

C₁-C₄-haloalkyl: a C₁-C₄-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl;

Cₗ-C₆-haloalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;

C₂-C₆-haloalkenyl: a C₂-C₆-alkenyl radical as mentioned above which is partially or fully substituted by fluorine, chlorine, bromine and/or iodine, for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromovinyl, 2-bromoallyl, 3-bromoallyl, 2,3-dibromoallyl, 3,3-dibromoallyl, 2,3,3-tribromoallyl or 2,3-dibromobut-2-enyl;

C₂-C₆-haloalkynyl: a C₂-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;

C₁-C₄-alkoxy and the alkoxy moieties of hydroxy-C₁-C₄-alkoxy-C₁-C₄-alkyl: for example methoxy, ethoxy, propoxy, 1-methylethoxy, butoxy, 1-methylpropoxy, 2-methylpropoxy and 1,1-dimethylethoxy;

C₁-C₆-alkoxy: C₁-C₄-alkoxy as mentioned above and also, for example, pentoxy, l-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

C₃-C₆-alkenyloxy: for example prop-1-en-1-yloxy, prop-2-en-1-yloxy, 1-methylethenyloxy, buten-1-yloxy, buten-2-yloxy, buten-3-yloxy, 1-methylprop-1-en-1-yloxy, 2-methylprop-1-en-1-yloxy, 1-methylprop-2-en-1-yloxy, 2-methylprop-2-en-1-yloxy, penten-1-yloxy, penten-2-yloxy, penten-3-yloxy, penten-4-yloxy, 1-methylbut-1-en-1-yloxy, 2-methylbut-1-en-1-yloxy, 3-methylbut-1-en-1-yloxy, 1-methylbut-2-en-1-yloxy, 2-methylbut-2-en-1-yloxy, 3-methylbut-2-en-1-yloxy, 1-methylbut-3-en-1-yloxy, 2-methylbut-3-en-1-yloxy, 3-methylbut-3-en-1-yloxy, 1,1-dimethylprop-2-en-1-yloxy, 1,2-dimethylprop-1-en-1-yloxy, 1,2-dimethylprop-2-en-1-yloxy, 1-ethylprop-1-en-2-yloxy, 1-ethylprop-2-en-1-yloxy, hex-1-en-1-yloxy, hex-2-en-1-yloxy, hex-3-en-1-yloxy, hex-4-en-1-yloxy, hex-5-en-1-yloxy, 1-methylpent-1-en-1-yloxy, 2-methylpent-1-en-1-yloxy, 3-methylpent-1-en-1-yloxy, 4-methylpent-1-en-1-yloxy, 1-methylpent-2-en-1-yloxy, 2-methylpent-2-en-1-yloxy, 3-methylpent-2-en-1-yloxy, 4-methylpent-2-en-1-yloxy,. 1-methylpent-3-en-1-yloxy, 2-methylpent-3-en-1-yloxy, 3-methylpent-3-en-1-yloxy, 4-methylpent-3-en-1-yloxy, 1-methylpent-4-en-1-yloxy, 2-methylpent-4-en-1-yloxy, 3-methylpent-4-en-1-yloxy, 4-methylpent-4-en-1-yloxy, 1,1-dimethylbut-2-en-1-yloxy, 1,1-dimethylbut-3-en-1-yloxy, 1,2-dimethylbut-1-en-1-yloxy, 1,2-dimethylbut-2-en-1-yloxy, 1,2-dimethylbut-3-en-1-yloxy, 1,3-dimethylbut-1-en-1-yloxy, 1,3-dimethylbut-2-en-1-yloxy, 1,3-dimethylbut-3-en-1-yloxy, 2,2-dimethylbut-3-en-1-yloxy, 2,3-dimethylbut-1-en-1-yloxy, 2,3-dimethylbut-2-en-1-yloxy, 2,3-dimethylbut-3-en-1-yloxy, 3,3-dimethylbut-1-en-1-yloxy, 3,3-dimethylbut-2-en-1-yloxy, 1-ethylbut-1-en-1-yloxy, 1-ethylbut-2-en-1-yloxy, 1-ethylbut-3-en-1-yloxy, 2-ethylbut-1-en-1-yloxy, 2-ethylbut-2-en-1-yloxy, 2-ethylbut-3-en-1-yloxy, 1,1,2-trimethylprop-2-en-1-yloxy, 1-ethyl-1-methylprop-2-en-1-yloxy, 1-ethyl-2-methylprop-1-en-1-yloxy and 1-ethyl-2-methylprop-2-en-1-yloxy;

C₃-C₆-alkynyloxy: for example prop-1-yn-1-yloxy, prop-2-yn-1-yloxy, but-1-yn-1-yloxy, but-1-yn-3-yloxy, but-1-yn-4-yloxy, but-2-yn-1-yloxy, pent-1-yn-1-yloxy, pent-1-yn-3-yloxy, pent-1-yn-4-yloxy, pent-1-yn-5-yloxy, pent-2-yn-1-yloxy, pent-2-yn-4-yloxy, pent-2-yn-5-yloxy, 3-methylbut-1-yn-3-yloxy, 3-methylbut-1-yn-4-yloxy, hex-1-yn-1-yloxy, hex-1-yn-3-yloxy, hex-1-yn-4-yloxy, hex-1-yn-5-yloxy, hex-1-yn-6-yloxy, hex-2-yn-1-yloxy, hex-2-yn-4-yloxy, hex-2-yn-5-yloxy, hex-2-yn-6-yloxy, hex-3-yn-1-yloxy, hex-3-yn-2-yloxy, 3-methylpent-1-yn-1-yloxy, 3-methylpent-1-yn-3-yloxy, 3-methylpent-1-yn-4-yloxy, 3-methylpent-1-yn-5-yloxy, 4-methylpent-1-yn-1-yloxy, 4-methylpent-2-yn-4-yloxy and 4-methylpent-2-yn-5-yloxy;

C₁-C₆-haloalkoxy: a C₁-C₆-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoropentoxy, 5-chloropentoxy, 5-bromopentoxy, 5-iodopentoxy, undecafluoropentoxy, 6-fluorohexoxy, 45 6-chlorohexoxy, 6-bromohexoxy, 6-iodohexoxy and dodecafluorohexoxy;

C₁-C₆-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methylpropoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(propoxy)propyl, 3-(1-methylethoxy)propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;

C₁-C₆-alkylamino: for example methylamino, ethylamino, propylamino, 1-methylethylamino, butylamino, 1-methylpropylamino, 2-methylpropylamino, 1,1-dimethylethylamino, pentylamino, 1-methylbutylamino,. 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino;

di(C₁-C₄-alkyl)amino: for example N,N-dimethylamino, N,N-diethylamino, N,N-dipropylamino, N,N-di(1-methylethyl)amino, N,N-dibutylamino, N,N-di(1-methylpropyl)amino, N,N-di(2-methylpropyl)amino, N,N-di(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino and N-(1,1-dimethylethyl)-N-(2-methylpropyl)amino;

C₁-C₆-alkylthio: for example methylthio, ethylthio, propylthio, 1-methylethylthio, butylthio, 1-methylpropylthio, 2-methylpropylthio and 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

C₁-C₆-haloalkylthio: a C₁-C₆-alkylthio radical as mentioned above which is partially or fully substituted by fluorine, * chlorine, bromine and/or iodine, i.e., for example, fluoromethylthio, difluoromethylthio, trifluoromethylthio, chlorodifluoromethylthio, bromodifluoromethylthio, 2-fluoroethylthio, 2-chloroethylthio, 2-bromoethylthio, 2-iodoethylthio, 2,2-difluoroethylthio, 2,2,2-trifluoroethylthio, 2,2,2-trichloroethylthio, 2-chloro2-fluoroethylthio, 2-chloro-2,2-difluoroethylthio, 2,2-dichloro-2-fluoroethylthio, pentafluoroethylthio, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2,3-dichloropropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, 4-bromobutylthio, nonafluorobutylthio, 5-flubropentylthio, 5-chloropentylthio, 5-bromopentylthio, 5-iodopentylthio, undecafluoropentylthio, 6-fluorohexylthio, 6-chlorohexylthio, 6-bromohexylthio, 6-iodohexylthio and dodecafluorohexylthio;

C-hd 1-C₆₁-alkylsulfinyl (C₁-C₆-alkyl-S(═O)—): for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;

C₁-C₆-haloalkylsulfinyl: a C₁-C₆-alkylsulfinyl radical as mentioned above which is partially or fully substituted by fluorine, chlorine, bromine and/or iodine, i.e., for example, fluoromethylsulfinyl, difluoromethylsulfinyl, trifluoromethylsulfinyl, chlorodifluoromethylsulfinyl, bromodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2,2,2-trichloroethylsulfinyl, 2-chloro-2-fluoroethylsulfinyl, 2-chloro-2,2-difluoroethylsulfinyl, 2,2-dichloro-2-fluoroethylsulfinyl, pentafluoroethylsulfinyl, 2-fluoropropylsulfinyl, 3-fluoropropylsulfinyl, 2-chloropropylsulfinyl, 3-chloropropylsulfinyl, 2-bromopropylsulfinyl, 3-bromopropylsulfinyl, 2,2-difluoropropylsulfinyl, 2,3-difluoropropylsulfinyl, 2,3-dichloropropylsulfinyl, 3,3,3-trifluoropropylsulfinyl, 3,3,3-trichloropropylsulfinyl, 2,2,3,3,3-pentafluoropropylsulfinyl, heptafluoropropylsulfinyl, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl, nonafluorobutylsulfinyl, 5-fluoropentylsulfinyl, 5-chloropentylsulfinyl, 5-bromopentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl and dodecafluorohexylsulfinyl;

C₁C-C₆-alkylsulfonyl (C₁-C₆-alkyl-S(═O)₂—): for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;

C₁C-C₆-haloalkylsulfonyl: a C₁-C₆-alkylsulfonyl radical as mentioned above which is partially or fully substituted by fluorine, chlorine, bromine and/or iodine, i.e., for example, fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-2chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6-fluorohexylsulfonyl, 6-bromohexylsulfonyl, 6-iodohexylsulfonyl and dodecafluorohexylsulfonyl.

In a particular embodiment, the variables of the compounds of the formula I have the following meanings, these meanings, both on their own and in combination with one another, being particular embodiments of the compounds of the formula I:

Preference is given to the N-heterocyclyl-substituted thienyloxypyrimidines of the formula I in which the variables W, X, Y and Z are CR³.

Preference is given to the N-heterocyclyl-substituted thienyloxypyrimidines of the formula I in which one or two of the variables W, X, Y, Z are N.

Preference is also given to the N-heterocyclyl-substituted thienyloxypyrimidines of the formula I in which one of the variables X or Z or two of the variables W and Z or X and Y are N.

Particular preference is given to the N-heterocyclyl-substituted thienyloxypyrimidines of the formula I in which two of the variables W, X, Y, Z, particularly preferably W and Z, are N.

Particular preference is also given to the N-heterocyclyl-substituted thienyloxypyrimidines of the formula I in which the variables W and Z, or W and X, or W and Y are N.

Particular preference is also given to the N-heterocyclyl-substituted thienyloxypyrimidines of the formula I in which one of the variables W, X, Y, Z, particularly preferably W or Z, is N.

Particular preference is also given to the N-heterocyclyl-substituted thienyloxypyrimidines of the formula I in which the variables X or Z are N.

Very-particular preference is given to the N-heterocyclyl-substituted thienyloxypyrimidines of the formula I in which the variables W, Y and Z are N or CH and X is CR³.

Particular preference is also given to the N-heterocyclyl-substituted thienyloxypyrimidines of the formula I in which the variables Y or Z are N and the variables W and X are CR³.

Particular preference is also given to the N-heterocyclyl-substituted thienyloxypyrimidines of the formula I in which the variable W is CH, Y is N or CH, Z is N or CH and X is CR³.

Preference is also given to the N-heterocyclyl-substituted thienyloxypyrimidines of the formula I in which the variable Y is N and W, X and Z are CR³.

Very particular preference is also given to the N-heterocyclyl-substituted thienyloxypyrimidines of the formula I in which W is N, X is CR³, Y is CH, Z is CH.

Very particular preference is also given to the N-heterocyclyl-substituted thienyloxypyrimidines of the formula I in which W is CH, X is N, Y is CR³, Z is CR³.

Very particular preference is also given to the N-heterocyclyl-substituted thienyloxypyrimidines of the formula I in which W is CR³, X is CR³, Y is CH, Z is N.

Preference is also given to the N-heterocyclyl-substituted thienyloxypyrimidines of the formula I in which

R¹ is hydrogen, halogen, cyano, C₁-C₆-alkyl, C₁-C₆-haloalkyl or C₁-C₆-alkoxy;

particularly preferably hydrogen, halogen such as fluorine, chlorine or bromine, C₁-C₆-alkyl such as methyl or ethyl;

with particular preference hydrogen, fluorine, chlorine or methyl.

Preference is also given to the N-heterocyclyl-substituted thienyloxypyrimidines of the formula I in which

R¹ is hydrogen, halogen, cyano, C₁-C₆-haloalkyl or C₁-C₆-alkoxy;

particularly preferably hydrogen, halogen, such as fluorine, chlorine or bromine;

with particular preference hydrogen, fluorine or chlorine.

Preference is also given to the N-heterocyclyl-substituted thienyloxypyrimidines of the formula I in which

R¹ is hydrogen or C₁-C₆-alkoxy, such as, for example, methoxy or ethoxy;

particularly preferably hydrogen or methoxy;

with particular preference methoxy.

Preference is also given to the N-heterocyclyl-substituted thienyloxypyrimidines of the formula I in which

R² is hydrogen, halogen, cyano, C₁-C₆-alkyl, Cₗ-C₆-haloalkyl, C₁-C₆-alkoxy, Cₗ-C₆-haloalkoxy, Cₗ-C₆-alkoxy-Cₗ-C₄-alkyl, C₁-C₆-alkylthio or COOR⁷;

particularly preferably hydrogen, halogen, cyano, C₁-C₆-alkyl, C₁-C₆-haloalkyl, C₁-C₆-alkoxy, C₁-C₆-alkoxy-C₁-C₄-alkyl or C₁-C₆-alkylthio;

with particular preference hydrogen, halogen such as fluorine, chlorine or bromine, C₁-C₆-alkyl such as methyl or ethyl, or C₁-C₆-haloalkyl such as trifluoromethyl, trichloromethyl or difluoromethyl;

very preferably hydrogen, fluorine, chlorine, methyl or trifluoromethyl.

Preference is also given to the N-heterocyclyl-substituted thienyloxypyrimidines of the formula I in which

R² is hydrogen, halogen, such as fluorine, chlorine or bromine, C₁-C₆-alkyl, such as methyl or ethyl, C₁-C₆-haloalkyl, such as trifluoromethyl, trichloromethyl or difluoromethyl, or C₁-C₆-alkoxy, such as methoxy or ethoxy;

particularly preferably hydrogen, fluorine, chlorine, methyl, trifluoromethyl or methoxy.

Preference is also given to the N-heterocyclyl-substituted thienyloxypyrimidines of the formula I in which

R² is halogen, cyano, Cₗ-C₆-haloalkyl, Cₗ-C₆-alkoxy, Cₗ-C₆-haloalkoxy, Cₗ-C₆-alkoxy-C₁-C₆-alkyl, C₁-C₆-alkylthio or COOR⁷;

particularly preferably halogen, cyano, C₁-C₆-haloalkyl, C₁-C₆-alkoxy, C₁-C₆-alkoxy-C₁-C₆-alkyl or C₁-C₆-alkylthio;

with particular preference halogen, such as fluorine, chlorine or bromine, C₁-C₆-haloalkyl, such as trifluoromethyl, trichloromethyl or difluoromethyl, or C₁-C₆-alkoxy, such as methoxy or ethoxy;

very preferably fluorine, chlorine, trifluoromethyl or methoxy.

Preference is also given to the N-heterocyclyl-substituted thienyloxypyrimidines of the formula I in which

R³ is hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl or COOR⁷;

particularly preferably hydrogen, halogen, cyano, C₁-C₆-alkyl, C₁-C₆-haloalkyl or C₁-C₆-alkoxy;

with particular preference hydrogen, halogen such as fluorine, chlorine or bromine, or C₁-C₆-haloalkyl such as trifluoromethyl, trichloromethyl or difluoromethyl;

very preferably hydrogen, fluorine, chlorine or trifluoromethyl.

Preference is also given to the N-heterocyclyl-substituted thienyloxypyrimidines of the formula I in which

R³ is hydrogen, halogen, cyano, C₁-C₆-alkyl, C₁-C₆-alkoxy, C₁-C₆-haloalkoxy, C₁-C₆-alkylthio, C₁-C₆-alkylsulfonyl or COOR⁷;

particularly preferably hydrogen, halogen, cyano, C₁-C₆-alkyl, C₁-C₆-alkoxy or C₁-C₆-haloalkoxy;

with particular preference hydrogen, halogen, such as fluorine, chlorine or bromine, or C₁-C₄-haloalkoxy, such as difluoromethoxy or trifluoromethoxy;

very preferably hydrogen, fluorine, chlorine, difluoromethoxy or trifluoromethoxy.

Preference is also given to the N-heterocyclyl-substituted thienyloxypyrimidines of the formula I in which in each-case independently of one another

R⁴, R⁵, R⁶ are hydrogen, halogen, cyano, C₁-C₆-alkyl, C₁-C₆-haloalkyl, C₁-C₆-alkoxy, Cₗ-C₆-haloalkoxy, Cₗ-C₆-alkylthio, Cₗ-C₆-alkylsulfonyl, Cₗ-C₆-haloalkylsulfonyl;

particularly preferably hydrogen, halogen, cyano, C₁-C₆-alkyl, Cₗ-C₆-haloalkyl, Cₗ-C₆-haloalkoxy, C₁-C₆-alkylsulfonyl or C₁-C₆-haloalkylsulfonyl;

with particular preference hydrogen, halogen such as fluorine, chlorine or bromine, Cₗ-C₆-haloalkyl such as trifluoromethyl, trichloromethyl or difluoromethyl, Cₗ-C₆-alkylsulfonyl such as methylsulfonyl or ethylsulfonyl, or C₁-C₆-haloalkylsulfonyl such as trifluoromethylsulfonyl, trichloromethylsulfonyl or difluoromethylsulfonyl;

very preferably hydrogen, fluorine, chlorine, trifluoromethyl or methylsulfonyl.

Preference is also given to the N-heterocyclyl-substituted thienyloxypyrimidines of the formula I in which in each case independently of one another

R⁴, R⁵, R⁶ are hydrogen, halogen, cyano, Cₗ-C₆-alkyl, Cₗ-C₆-alkoxy, C₁-C₆-haloalkoxy, C₁-C₆-alkylthio, C₁-C₆-alkylsulfonyl, C₁-C₆-haloalkylsulfonyl;

particularly preferably hydrogen, halogen, cyano, C₁-C₆-alkyl, C₁-C₆-haloalkoxy, C₁-C₆-alkylsulfonyl or C₁-C₆-haloalkylsulfonyl;

with particular preference hydrogen, halogen, such as fluorine, chlorine or bromine, Cₗ-C₆-haloalkoxy, such as difluoromethoxy or trifluoromethoxy, Cₗ-C₆-alkylsulfonyl, such as methylsulfonyl or ethylsulfonyl, or Cₗ-C₆-haloalkylsulfonyl, such as trifluoromethylsulfonyl, trichloromethylsulfonyl or difluoromethylsulfonyl;

very preferably hydrogen, fluorine, chlorine, difluoromethoxy, trifluoromethoxy or methylsulfonyl.

Preference is also given to the N-heterocyclyl-substituted thienyloxypyrimidines of the formula I in which R⁶ is hydrogen and, in each case independently of one another,

R⁴, R⁵ are hydrogen, halogen, C₁-C₆-alkyl or C₁-C₆-haloalkyl;

particularly preferably hydrogen, chlorine, methyl or trifluoromethyl.

Particular preference is also given to the N-heterocyclyl-substituted thienyloxypyrimidines of the formula I in which the thienyl radical is attached in the 3-position via the oxygen atom with the pyrimidine skeleton and is substituted by R⁴ and R⁵ in positions 4 and 5.

Particular preference is also given to the N-heterocyclyl-substituted thienyloxypyrimidines of the formula I in which the thienyl radical is attached in the 2-position via the oxygen atom to the pyrimidine skeleton and is substituted by R⁴ and R⁵ in positions 4 and 5.

Preference is also given to the N-heterocyclyl-substituted thienyloxypyrimidines of the formula I in which R⁵ and R⁶ are hydrogen and

R⁴ is halogen, cyano, C₁-C₆-alkyl, C₁-C₆-haloalkyl or Cₗ-C₆-alkoxy;

particularly-preferably halogen or C₁-C₆-haloalkyl;

very preferably fluorine, chlorine or trifluoromethyl.

Preference is also given to the N-heterocyclyl-substituted thienyloxypyrimidines of the formula I in which R⁵ and R⁶ are hydrogen and

R⁴ is halogen, cyano, C₁-C₆-alkyl or C₁-C₆-alkoxy; particularly preferably halogen;

very preferably fluorine or chlorine.

Particular preference is also given to the N-heterocyclyl-substituted thienyloxypyrimidines of the formula I in which the thienyl radical is attached in the 3-position via the oxygen atom to the pyrimidine skeleton and is-substituted by R⁴ in position 5.

Particular preference is also given to the N-heterocyclyl-substituted thienyloxypyrimidines of the formula I in which the thienyl radical is attached in the 3-position via the oxygen atom to the pyrimidine skeleton, R⁵ and R⁶ are hydrogen and the thienyl radical in the 5-position is substituted by R⁴, where

R⁴ is halogen, cyano, C₁-C₆-alkyl, trichloromethyl, difluoromethyl, monofluoromethyl, C₁-C₆-alkoxy or C₁-C₆-haloalkoxy;

particularly preferably halogen, trichloromethyl, difluoromethyl, monofluoromethyl, difluoromethoxy or trifluoromethoxy;

very preferably fluorine, chlorine, trichloromethyl, difluoromethyl, monofluoromethyl, difluoromethoxy or trifluoromethoxy.

Particular preference is also given to the N-heterocyclyl-substituted thienyloxypyrimidines of the formula I in which the, thienyl radical is attached in the 2-position via the oxygen atom to the pyrimidine skeleton and is substituted by R⁴ in position 5.

Preference is also given to the N-heterocyclyl-substituted thienyloxypyrimidines of the formula I in which

R¹ is hydrogen or C₁-C₆-alkyl;

R² is hydrogen, halogen, cyano, C₁-C₆-alkyl, C₁-C₆-haloalkyl, C₁-C₆-alkoxy, C₁-C₆-alkylthio, Cₗ-C₆-alkoxy-Cₗ-C₄-alkyl or COOR⁷.

Preference is also given to the N-heterocyclyl-substituted thienyloxypyrimidines of the formula I in which

R¹ is hydrogen; and

R² is halogen, cyano, C₁-C₆-haloalkyl, C₁-C₆-alkoxy, C₁-C₆-alkylthio, C₁-C₆-alkoxy-C₁-C₄-alkyl or COOR⁷.

Particular preference is also given to the N heterocyclyl-substituted thienyloxypyrimidines of the formula I in which

R¹ is C₁-C₆-alkoxy;

particularly preferably C₁-C₄-alkoxy, such as, for example, methoxy or ethoxy;

with particular preference methoxy; and

R² is hydrogen.

Particular preference is also given to the N-heterocyclyl-substituted thienyloxypyrimidines of the formula I in which

R¹ is hydrogen or C₁-C₆-alkoxy;

particularly preferably hydrogen, or Cₗ-C₄-alkoxy, such as, for example, methoxy or ethoxy;

with particular preference hydrogen or methoxy; and

R² is hydrogen.

Preference is also given to the N-heterocyclyl-substituted thienyloxypyrimidines of the formula I in which the thienyl radical in the 3-position is attached via the oxygen atom to the pyrimidine skeleton and the variables Y or Z are N and W and X are CR³.

Preference is also given to the N-heterocyclyl-substituted thienyloxypyrimidines of the formula I in which the thienyl radical in the 3-position is attached via the oxygen atom to the pyrimidine skeleton and the variable W is CR³, Y is N or CR³, Z is N or CR³ and X is CR³.

Particular preference is also given to the N-heterocyclyl-substituted thienyloxypyrimidines of the formula I in which the thienyl radical in the 3-position is attached via the oxygen atom to the pyrimidine skeleton and is substituted by R⁴ in position 5, where

R¹ is hydrogen, Cₗ-C₆-alkyl, such as, for example, methyl, or C₁-C₆-alkoxy, such as, for example, methoxy, particularly preferably hydrogen, methyl or methoxy, with particular-preference methyl;

R² is hydrogen;

R⁴ is C₁-C₆-haloalkyl, particularly preferably trifluoromethyl or difluoromethyl;

R⁵, R⁶ are hydrogen;

and the variable W is CR³, X is CR³, Y is CH, and Z is N; where

R³ is hydrogen or halogen, preferably hydrogen or chlorine, with particular preference chlorine.

Most preference is given to compounds of the formula Ia (where X═C—CF₃, R⁴=5-CF₃, R⁵═H, R⁶═H; the thienyl radical is attached in the 3-position via an oxygen atom to the pyrimidine skeleton), in particular to the compounds Ia.1 to Ia.121 of Table 1, where the definitions of the variables W, Y, Z, R¹ and R² play a particular role for the compounds according to the invention, not only in combination with one another but in each case also on their own.

TABLE 1

| | |
|---|---|
| | Ia |
| 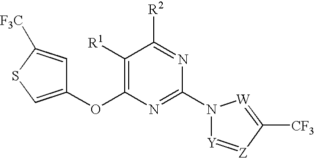 | |

| No. | R¹ | R² | W | Y | Z |
|---|---|---|---|---|---|
| Ia.1 | H | H | CH | CH | CH |
| Ia.2 | H | H | N | CH | CH |
| Ia.3 | H | H | CH | N | CH |
| Ia.4 | H | H | CH | CH | N |
| Ia.5 | H | H | N | CH | N |
| Ia.6 | H | H | N | N | N |
| Ia.7 | CH₃ | CH₃ | CH | CH | CH |
| Ia.8 | CH₃ | CH₃ | N | CH | CH |
| Ia.9 | CH₃ | CH₃ | CH | N | CH |
| Ia.10 | CH₃ | CH₃ | CH | CH | N |
| Ia.11 | CH₃ | CH₃ | N | CH | N |
| Ia.12 | CH₃ | CH₃ | N | N | N |
| Ia.13 | H | OCH₃ | CH | CH | CH |
| Ia.14 | H | OCH₃ | N | CH | CH |
| Ia.15 | H | OCH₃ | CH | N | CH |
| Ia.16 | H | OCH₃ | CH | CH | N |
| Ia.17 | H | OCH₃ | N | CH | N |
| Ia.18 | H | OCH₃ | N | N | N |
| Ia.19 | H | CN | CH | CH | CH |
| Ia.20 | H | CN | N | CH | CH |
| Ia.21 | H | CN | CH | N | CH |
| Ia.22 | H | CN | CH | CH | N |
| Ia.23 | H | CN | N | CH | N |
| Ia.24 | H | CN | N | N | N |
| Ia.25 | H | SCH₃ | CH | CH | CH |
| Ia.26 | H | SCH₃ | N | CH | CH |
| Ia.27 | H | SCH₃ | CH | N | CH |
| Ia.28 | H | SCH₃ | CH | CH | N |
| Ia.29 | H | SCH₃ | N | CH | N |
| Ia.30 | H | SCH₃ | N | N | N |
| Ia.31 | H | CF₃ | CH | CH | CH |
| Ia.32 | H | CF₃ | N | CH | CH |
| Ia.33 | H | CF₃ | CH | N | CH |
| Ia.34 | H | CF₃ | CH | CH | N |
| Ia.35 | H | CF₃ | N | CH | N |
| Ia.36 | H | CF₃ | N | N | N |
| Ia.37 | H | Cl | CH | CH | CH |
| Ia.38 | H | Cl | N | CH | CH |
| Ia.39 | H | Cl | CH | N | CH |
| Ia.40 | H | Cl | CH | CH | N |
| Ia.41 | H | Cl | N | CH | N |
| Ia.42 | H | Cl | N | N | N |
| Ia.43 | H | Br | CH | CH | CH |
| Ia.44 | H | Br | N | CH | CH |
| Ia.45 | H | Br | CH | N | CH |
| Ia.46 | H | Br | CH | CH | N |
| Ia.47 | H | Br | N | CH | N |
| Ia.48 | H | Br | N | N | N |
| Ia.49 | H | F | CH | CH | CH |
| Ia.50 | H | F | N | CH | CH |
| Ia.51 | H | F | CH | N | CH |
| Ia.52 | H | F | CH | CH | N |
| Ia.53 | H | F | N | CH | N |
| Ia.54 | H | F | N | N | N |
| Ia.55 | H | CH₂OCH₃ | CH | CH | CH |
| Ia.56 | H | CH₂OCH₃ | N | CH | CH |
| Ia.57 | H | CH₂OCH₃ | CH | N | CH |
| Ia.58 | H | CH₂OCH₃ | CH | CH | N |
| Ia.59 | H | CH₂OCH₃ | N | CH | N |
| Ia.60 | H | CH₂OCH₃ | N | N | N |
| Ia.61 | H | CO₂C₂H₅ | CH | CH | CH |
| Ia.62 | H | CO₂C₂H₅ | N | CH | CH |
| Ia.63 | H | CO₂C₂H₅ | CH | N | CH |
| Ia.64 | H | CO₂C₂H₅ | CH | CH | N |
| Ia.65 | H | CO₂C₂H₅ | N | CH | N |
| Ia.66 | H | CO₂C₂H₅ | N | N | N |
| Ia.67 | CH₃ | H | CH | CH | CH |
| Ia.68 | CH₃ | H | N | CH | CH |
| Ia.69 | CH₃ | H | CH | N | CH |
| Ia.70 | CH₃ | H | CH | CH | N |
| Ia.71 | CH₃ | H | N | CH | N |
| Ia.72 | CH₃ | H | N | N | N |
| Ia.73 | H | CH₃ | CH | CH | CH |
| Ia.74 | H | CH₃ | N | CH | CH |
| Ia.75 | H | CH₃ | CH | N | CH |
| Ia.76 | H | CH₃ | CH | CH | N |
| Ia.77 | H | CH₃ | N | CH | N |
| Ia.78 | H | CH₃ | N | N | N |
| Ia.79 | C₂H₅ | H | CH | CH | CH |
| Ia.80 | C₂H₅ | H | N | CH | CH |
| Ia.81 | C₂H₅ | H | CH | N | CH |
| Ia.82 | C₂H₅ | H | CH | CH | N |
| Ia.83 | C₂H₅ | H | N | CH | N |
| Ia.84 | C₂H₅ | H | N | N | N |
| Ia.85 | CF₃ | H | CH | CH | CH |
| Ia.86 | CF₃ | H | N | CH | CH |
| Ia.87 | CF₃ | H | CH | N | CH |
| Ia.88 | CF₃ | H | CH | CH | N |
| Ia.89 | CF₃ | H | N | CH | N |
| Ia.90 | CF₃ | H | N | N | N |
| Ia.91 | Cl | H | CH | CH | CH |
| Ia.92 | Cl | H | N | CH | CH |
| Ia.93 | Cl | H | CH | N | CH |
| Ia.94 | Cl | H | CH | CH | N |
| Ia.95 | Cl | H | N | CH | N |
| Ia.96 | Cl | H | N | N | N |
| Ia.97 | H₃CO | H | CH | CH | CH |
| Ia.98 | H₃CO | H | N | CH | CH |
| Ia.99 | H₃CO | H | CH | N | CH |
| Ia.100 | H₃CO | H | CH | CH | N |
| Ia.101 | H₃CO | H | N | CH | N |
| Ia.102 | H₃CO | H | N | N | N |
| Ia.103 | H | H | N | N | CH |
| Ia.104 | CH₃ | CH₃ | N | N | CH |
| Ia.105 | H | OCH₃ | N | N | CH |
| Ia.106 | H | CN | N | N | CH |
| Ia.107 | H | SCH₃ | N | N | CH |
| Ia.108 | H | CF₃ | N | N | CH |
| Ia.109 | H | Cl | N | N | CH |
| Ia.110 | H | Br | N | N | CH |
| Ia.111 | H | F | N | N | CH |
| Ia.112 | H | CH₂OCH₃ | N | N | CH |
| Ia.113 | H | CO₂C₂H₅ | N | N | CH |
| Ia.114 | CH₃ | H | N | N | CH |
| Ia.115 | H | CH₃ | N | N | CH |
| Ia.116 | C₂H₅ | H | N | N | CH |
| Ia.117 | CF₃ | H | N | N | CH |
| Ia.118 | Cl | H | N | N | CH |
| Ia.119 | H₃CO | H | N | N | CH |
| Ia.120 | H | H | CH | N | CCH₃ |
| Ia.121 | H | H | CCl | CH | N |

Most preference is also given to the compounds of the formula Ib, in particular to the compounds Ib.1 to Ib.121 which differ from the corresponding compounds Ia.1 to Ia.121 in that R⁴ is chlorine.

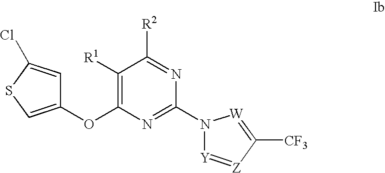

Most preference is also given to the compounds of the formula Ic, in particular to the compounds Ic.1 to Ic.121 which differ from the corresponding compounds Ia.1 to Ia.121 in that the thienyl radical is attached in the 2-position via the oxygen atom to the pyrimidine skeleton.

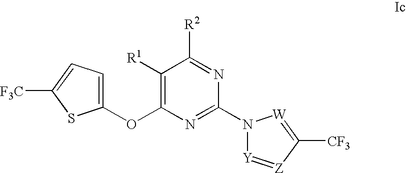

Most preference is also given to the compounds of the formula Id, in particular to the compounds Id.1 to-Id.121 which differ from the corresponding compounds Ia.1 to Ia.121 in that R⁴ is chlorine and the thienyl radical is attached in the 2-position via the oxygen atom to the pyrimidine skeleton.

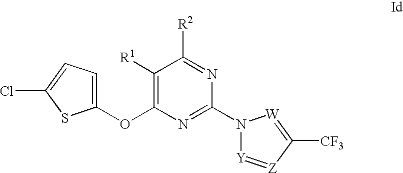

Most preference is also given to the compounds of the formula Ie, in particular to the compounds Ie.1 to Ie.121 which differ from the corresponding compounds Ia.1 to Ia.121 in that R⁴ imposition 5 and R⁵ in position 4 are chlorine.

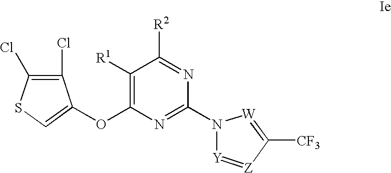

Most preference is also given to the compounds of the formula If, in particular to the compounds If.1 to If.121 which differ from the corresponding compounds Ia.1 to Ia.121 in that R⁴ in position 5 and R⁵ in position 2 are chlorine.

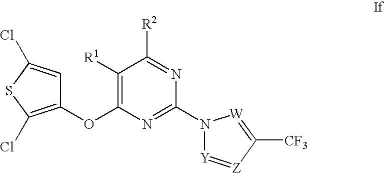

Most preference is also given to the compounds of the formula Ig, in particular to the compounds Ig.1 to Ig.121 which differ from the corresponding compounds Ia.1 to Ia.121 in that R⁴ in position 5 and R⁵ in position 4 are chlorine and the thienyl radical is attached in the 2-position via the oxygen atom to the pyrimidine skeleton.

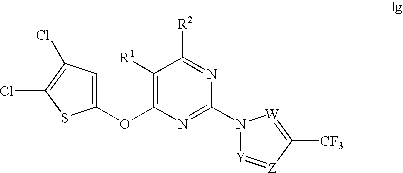

Most preference is also given to the compounds of the formula Ih, in particular to the compounds Ih.1 to Ih.121 which differ from the corresponding compounds Ia.1 to Ia.121 in that X is C—NO₂.

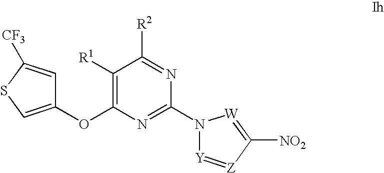

Most preference is also given to the compounds of the formula Ii, in particular to the compounds Ii.1 to Ii.121 which differ from the corresponding compounds Ia.1 to Ia.121 in that X is C—Cl.

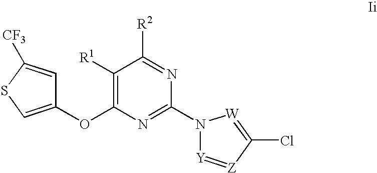

Most preference is also given to the compounds of the formula Ik, in particular to the compounds Ik.1 to Ik.121 which differ from the corresponding compounds Ia.1 to Ia.121 in that X is NO₂ and R⁴ is chlorine.

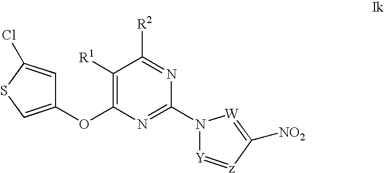

Most preference is also given to the compounds of the formula Il, 25 in particular to the compounds Il.1 to Il.121 which differ from the corresponding compounds Ia.1 to Ia.121 in that X is C—Cl and R⁴ is chlorine.

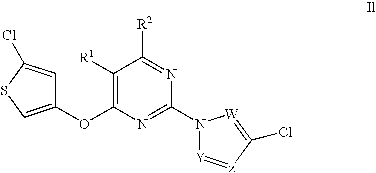

Most preference is also given to the compounds of the formula Im, in particular to the compounds Im.1 to Im.121 which differ from 40 the corresponding compounds Ia.1 to Ia.121 in that X is C—NO₂ and the thienyl radical in the 2-position is attached via the oxygen atom to the pyrimidine skeleton.

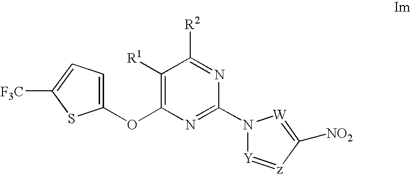

Most preference is also given to the compounds of the formula In, in particular to the compounds In.1 to In.121 which differ from the corresponding compounds Ia.1 to Ia.121 in that X is C—Cl and the thienyl radical in the 2-position is attached via the oxygen atom to the pyrimidine skeleton.

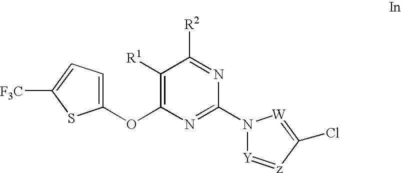

Most preference is also given to the compounds of the formula Io, in particular to the compounds Io.1 to Io.121 which differ from the corresponding compounds Ia.1 to Ia.121 in that X is C—NO₂, R⁴ is chlorine and the thienyl radical in the 2-position is attached via the oxygen atom to the pyrimidine skeleton.

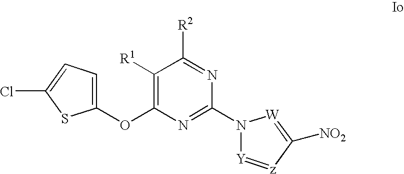

Most preference is also given to the compounds of the formula Ip, in particular to the compounds Ip.1 to Ip.121 which differ from the corresponding compounds Ia.1 to Ia.121 in that X is C—Cl, R⁴ is chlorine and the thienyl radical in the 2-position is attached via the oxygen atom to the pyrimidine skeleton.

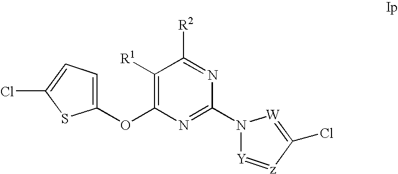

Preference is also given to the compounds of the formula Iq (where W is C—CR³, R⁴ is 5-CF₃, R⁵ is H, R⁶ is H; the thienyl radical is attached in the 3-position via an oxygen atom to the pyrimidine skeleton), in particular to the compounds Iq.1 to Iq.205 of table 2, where the definitions of the variables X, Y, Z, R¹, R² and R³ play a particular role for the compounds according to the invention, not only in combination with one another but in each case also on their own.

TABLE 2

| | |
|---|---|
| | Iq |
| 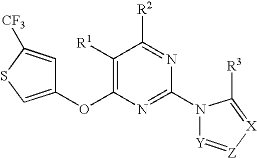 | |

| No. | R¹ | R² | R³ | X | Y | Z |
|---|---|---|---|---|---|---|
| Iq.1 | H | H | CF₃ | CH | CH | CH |
| Iq.2 | H | H | CF₃ | CH | N | CH |
| Iq.3 | H | H | CF₃ | CH | CH | N |
| Iq.4 | H | H | CF₃ | N | CH | CH |
| Iq.5 | H | H | CF₃ | N | CH | N |
| Iq.6 | H | H | CF₃ | CH | N | N |
| Iq.7 | H | H | CF₃ | N | N | CH |
| Iq.8 | H | H | CF₃ | N | N | N |
| Iq.9 | H | H | CN | CH | CH | CH |
| Iq.10 | H | H | CN | CH | N | CH |
| Iq.11 | H | H | CN | CH | CH | N |
| Iq.12 | H | H | CN | N | CH | CH |
| Iq.13 | H | H | CN | N | CH | N |
| Iq.14 | H | H | CN | CH | N | N |
| Iq.15 | H | H | CN | N | N | CH |
| Iq.16 | H | H | CN | N | N | N |
| Iq.17 | H | H | C(═O)CF₃ | CH | CH | CH |
| Ig.18 | H | H | C(═O)CF₃ | CH | N | CH |
| Iq.19 | H | H | C(═O)CF₃ | CH | CH | N |
| Iq.20 | H | H | C(═O)CF₃ | N | CH | CH |
| Iq.21 | H | H | C(═O)CF₃ | N | CH | N |
| Iq.22 | H | H | C(═O)CF₃ | CH | N | N |
| Iq.23 | H | H | C(═O)CF₃ | N | N | CH |
| Iq.24 | H | H | C(═O)CF₃ | N | N | N |
| Iq.25 | H | H | CH₃ | CH | CCH₃ | CH |
| Iq.26 | H | H | CH₃ | N | CCH₃ | N |
| Iq.27 | H | H | CH₃ | N | CCH₃ | CH |
| Iq.28 | H | H | Cl | CH | CCl | CH |
| Iq.29 | H | H | Cl | N | CCl | N |
| Iq.30 | H | H | Cl | N | CCl | CH |
| Iq.31 | H | H | Br | C-tC₄H₉ | N | N |
| Iq.32 | H | H | Br | C-tC₄H₉ | CH | CH |
| Iq.33 | H | H | Br | C-tC₄H₉ | CH | N |
| Iq.34 | CH₃ | CH₃ | CF₃ | CH | CH | CH |
| Iq.35 | CH₃ | CH₃ | CF₃ | CH | N | CH |
| Iq.36 | CH₃ | CH₃ | CF₃ | CH | CH | N |
| Iq.37 | CH₃ | CH₃ | CF₃ | N | CH | CH |
| Iq.38 | CH₃ | CH₃ | CF₃ | N | CH | N |
| Iq.39 | CH₃ | CH₃ | CF₃ | CH | N | N |
| Iq.40 | CH₃ | CH₃ | CF₃ | N | N | CH |
| Iq.41 | CH₃ | CH₃ | CF₃ | N | N | N |
| Iq.42 | CH₃ | CH₃ | CN | CH | CH | CH |
| Iq.43 | CH₃ | CH₃ | CN | CH | N | CH |
| Iq.44 | CH₃ | CH₃ | CN | CH | CH | N |
| Iq.45 | CH₃ | CH₃ | CN | N | CH | CH |
| Iq.46 | CH₃ | CH₃ | CN | N | CH | N |
| Iq.47 | CH₃ | CH₃ | CN | CH | N | N |
| Iq.48 | CH₃ | CH₃ | CN | N | N | CH |
| Iq.49 | CH₃ | CH₃ | CN | N | N | N |
| Iq.50 | CH₃ | CH₃ | C(═O)CF₃ | CH | CH | CH |
| Iq.51 | CH₃ | CH₃ | C(═O)CF₃ | CH | N | CH |
| Iq.52 | CH₃ | CH₃ | C(═O)CF₃ | CH | CH | N |
| Iq.53 | CH₃ | CH₃ | C(═O)CF₃ | N | CH | CH |
| Iq.54 | CH₃ | CH₃ | C(═O)CF₃ | N | CH | N |
| Iq.55 | CH₃ | CH₃ | C(═O)CF₃ | CH | N | N |
| Iq.56 | CH₃ | CH₃ | C(═O)CF₃ | N | N | CH |
| Iq.57 | CH₃ | CH₃ | C(═O)CF₃ | N | N | N |
| Iq.58 | CH₃ | CH₃ | CH₃ | CH | CCH₃ | CH |
| Iq.59 | CH₃ | CH₃ | CH₃ | N | CCH₃ | N |
| Iq.60 | CH₃ | CH₃ | CH₃ | N | CCH₃ | CH |
| Iq.61 | CH₃ | CH₃ | Cl | CH | CCl | CH |
| Iq.62 | CH₃ | CH₃ | Cl | N | CCl | N |
| Iq.63 | CH₃ | CH₃ | Cl | N | CCl | CH |
| Iq.64 | CH₃ | CH₃ | Br | C-tC₄H₉ | N | N |
| Iq.65 | CH₃ | CH₃ | Br | C-tC₄H₉ | CH | CH |
| Iq.66 | CH₃ | CH₃ | Br | C-tC₄H₉ | CH | N |
| Ig.67 | CH₃ | H | CF₃ | CH | CH | CH |
| Iq.68 | CH₃ | H | CF₃ | CH | N | CH |
| Iq.69 | CH₃ | H | CF₃ | CH | CH | N |
| Iq.70 | CH₃ | H | CF₃ | N | CH | CH |
| Iq.71 | CH₃ | H | CF₃ | N | CH | N |
| Iq.72 | CH₃ | H | CF₃ | CH | N | N |
| Iq.73 | CH₃ | H | CF₃ | N | N | CH |
| Iq.74 | CH₃ | H | CF₃ | N | N | N |
| Iq.75 | CH₃ | H | CN | CH | CH | CH |
| Iq.76 | CH₃ | H | CN | CH | N | CH |
| Iq.77 | CH₃ | H | CN | CH | CH | N |
| Iq.78 | CH₃ | H | CN | N | CH | CH |
| Iq.79 | CH₃ | H | CN | N | CH | N |
| Iq.80 | CH₃ | H | CN | CH | N | N |
| Ig.81 | CH₃ | H | CN | N | N | CH |
| Iq.82 | CH₃ | H | CN | N | N | N |
| Iq.83 | CH₃ | H | C(═O)CF₃ | CH | CH | CH |
| Iq.84 | CH₃ | H | C(═O)CF₃ | CH | N | CH |
| Iq.85 | CH₃ | H | C(═O)CF₃ | CH | CH | N |
| Iq.86 | CH₃ | H | C(═O)CF₃ | N | CH | CH |
| Iq.87 | CH₃ | H | C(═O)CF₃ | N | CH | N |
| Iq.88 | CH₃ | H | C(═O)CF₃ | CH | N | N |
| Iq.89 | CH₃ | H | C(═O)CF₃ | N | N | CH |
| Iq.90 | CH₃ | H | C(═O)CF₃ | N | N | N |
| Iq.91 | CH₃ | H | CH₃ | CH | CCH₃ | CH |
| Iq.92 | CH₃ | H | CH₃ | N | CCH₃ | N |
| Iq.93 | CH₃ | H | CH₃ | N | CCH₃ | CH |
| Iq.94 | CH₃ | H | Cl | CH | CCl | CH |
| Iq.95 | CH₃ | H | Cl | N | CCl | N |
| Iq.96 | CH₃ | H | Cl | N | CCl | CH |
| Iq.97 | CH₃ | H | Br | C-tC₄H₉ | N | N |
| Iq.98 | CH₃ | H | Br | C-tC₄H₉ | CH | CH |
| Ig.99 | CH₃ | H | Br | C-tC₄H₉ | CH | N |
| Iq.100 | H | CH₃ | CF₃ | CH | CH | CH |
| Iq.101 | H | CH₃ | CF₃ | CH | N | CH |
| Iq.102 | H | CH₃ | CF₃ | CH | CH | N |
| Iq.103 | H | CH₃ | CF₃ | N | CH | CH |
| Iq.104 | H | CH₃ | CF₃ | N | CH | N |
| Iq.105 | H | CH₃ | CF₃ | CH | N | N |
| Iq.106 | H | CH₃ | CF₃ | N | N | CH |
| Iq.107 | H | CH₃ | CF₃ | N | N | N |
| Iq.108 | H | CH₃ | CN | CH | CH | CH |
| Iq.109 | H | CH₃ | CN | CH | N | CH |
| Iq.110 | H | CH₃ | CN | CH | CH | N |
| Ig.111 | H | CH₃ | CN | N | CH | CH |
| Iq.112 | H | CH₃ | CN | N | CH | N |
| Iq.113 | H | CH₃ | CN | CH | N | N |
| Iq.114 | H | CH₃ | CN | N | N | CH |
| Iq.115 | H | CH₃ | CN | N | N | N |
| Iq.116 | H | CH₃ | C(═O)CF₃ | CH | CH | CH |
| Iq.117 | H | CH₃ | C(═O)CF₃ | CH | N | CH |
| Iq.118 | H | CH₃ | C(═O)CF₃ | CH | CH | N |
| Iq.119 | H | CH₃ | C(═O)CF₃ | N | CH | CH |
| Iq.120 | H | CH₃ | C(═O)CF₃ | N | CH | N |
| Iq.121 | H | CH₃ | C(═O)CF₃ | CH | N | N |
| Iq.122 | H | CH₃ | C(═O)CF₃ | N | N | CH |
| Iq.123 | H | CH₃ | C(═O)CF₃ | N | N | N |
| Iq.124 | H | CH₃ | CH₃ | CH | CCH₃ | CH |
| Iq.125 | H | CH₃ | CH₃ | N | CCH₃ | N |
| Iq.126 | H | CH₃ | CH₃ | N | CCH₃ | CH |
| Iq.127 | H | CH₃ | Cl | CH | CCl | CH |
| Iq.128 | H | CH₃ | Cl | N | CCl | N |
| Iq.129 | H | CH₃ | Cl | N | CCl | CH |
| Iq.130 | H | CH₃ | Br | C-tC₄H₉ | N | N |
| Iq.131 | H | CH₃ | Br | C-tC₄H₉ | CH | CH |
| Iq.132 | H | CH₃ | Br | C-tC₄H₉ | CH | N |
| Iq.133 | OCH₃ | H | CF₃ | CH | CH | CH |
| Iq.134 | OCH₃ | H | CF₃ | CH | N | CH |
| Iq.135 | OCH₃ | H | CF₃ | CH | CH | N |
| Iq.136 | OCH₃ | H | CF₃ | N | CH | CH |
| Iq.137 | OCH₃ | H | CF₃ | N | CH | N |
| Iq.138 | OCH₃ | H | CF₃ | CH | N | N |
| Iq.139 | OCH₃ | H | CF₃ | N | N | CH |
| Iq.140 | OCH₃ | H | CF₃ | N | N | N |
| Iq.141 | OCH₃ | H | CN | CH | CH | CH |
| Iq.142 | OCH₃ | H | CN | CH | N | CH |
| Iq.143 | OCH₃ | H | CN | CH | CH | N |
| Iq.144 | OCH₃ | H | CN | N | CH | CH |
| Iq.145 | OCH₃ | H | CN | N | CH | N |
| Iq.146 | OCH₃ | H | CN | CH | N | N |
| Iq.147 | OCH₃ | H | CN | N | N | CH |
| Iq.148 | OCH₃ | H | CN | N | N | N |
| Iq.149 | OCH₃ | H | C(═O)CF₃ | CH | CH | CH |
| Iq.150 | OCH₃ | H | C(═O)CF₃ | CH | N | CH |
| Iq.151 | OCH₃ | H | C(═O)CF₃ | CH | CH | N |
| Iq.152 | OCH₃ | H | C(═O)CF₃ | N | CH | CH |
| Iq.153 | OCH₃ | H | C(═O)CF₃ | N | CH | N |
| Iq.154 | OCH₃ | H | C(═O)CF₃ | CH | N | N |
| Iq.155 | OCH₃ | H | C(═O)CF₃ | N | N | CH |
| Iq.156 | OCH₃ | H | C(═O)CF₃ | N | N | N |
| Iq.157 | OCH₃ | H | CH₃ | CH | CCH₃ | CH |
| Iq.158 | OCH₃ | H | CH₃ | N | CCH₃ | N |
| Iq.159 | OCH₃ | H | CH₃ | N | CCH₃ | CH |
| Iq.160 | OCH₃ | H | Cl | CH | CCl | CH |
| Iq.161 | OCH₃ | H | Cl | N | CCl | N |
| Iq.162 | OCH₃ | H | Cl | N | CCl | CH |
| Iq.163 | OCH₃ | H | Br | C-tC₄H₉ | N | N |
| Iq.164 | OCH₃ | H | Br | C-tC₄H₉ | CH | CH |
| Iq.165 | OCH₃ | H | Br | C-tC₄H₉ | CH | N |

Most preference is also given to the compounds of the formula Ir, in particular to the compounds Ir.1 to Ir.165 which differ from the corresponding compounds Iq.1 to Iq.165 in that R⁴ is chlorine.

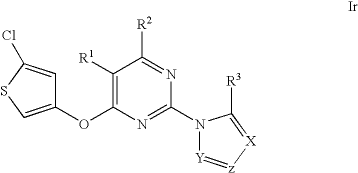

Most preference is also given to the compounds of the formula Is, in particular to the compounds Is.1 to Is.165 which differ from the corresponding compounds Iq.1 to Iq.165 in that the thienyl radical is attached in the 2-position via the oxygen atom to the pyrimidine skeleton.

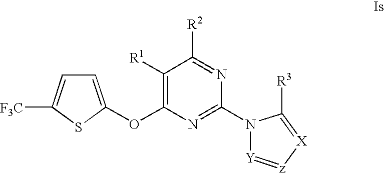

Most preference is also given to the compounds of the formula It, 30in particular to the compounds It.1 to It.165 which differ from the corresponding compounds Iq.1 to Iq.165 in that R⁴ is chlorine and the thienyl radical is attached in the 2-position via the oxygen atom to the pyrimidine skeleton.

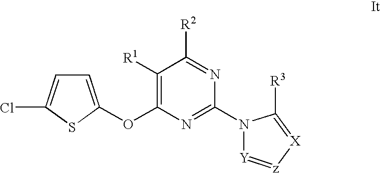

The N-heterocyclyl-substituted thienyloxypyrimidines of the formula I can be obtained by various methods, for example by the processes below.

Process A

Dihaloheterocycles of the formula VIII are obtained, for example, from dicarbonylheterocycles by reaction with a chlorinating agent. Dichloroheterocycles of the formula VIII can also be obtained commercially.

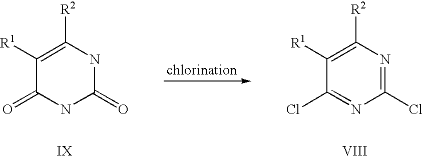

This reaction is usually carried out at temperatures of from 25° C. to 130° C. in the presence of a base [cf. Advances in Heterocyclic Chemistry, Ed. A. R. Katritzky, 1993, 58, 301-305; Heterocyclic Compounds, Ed. R. C. Ederfield, 1057, 6, 265-270].

Suitable chlorinating agents are, for example, phosphorus oxychloride, neat or in the presence of a solvent, or sulfuryl chloride.

Suitable bases are, in general, organic bases, for example tertiary amines such as trimethylamine, triethylamine, diisopropylethylamine, N,N-dimethylaniline and N-methylpiperidine, pyridine, substituted pyridines such as collidine, lutidine and 4-dimethylaminopyridine, and also bicyclic amines. Particular preference is given to N,N-dimethylaniline.

In general, the bases can be employed in catalytic amounts; however, they can also be employed in equimolar amounts, in excess or, if appropriate, as solvent.

The starting materials are generally reacted with one another in equimolar amounts. It may be advantageous to employ an excess of chlorinating agent, based on IX.

The starting materials required for preparing the compounds I are known from the literature, can be prepared similarly to methods known from the literature (E. Larsen et al., Synthesis 8 (1995), 934-936; JP-56139467; Organic Synthesis II (1943), 422), or they are commercially available.

The dihaloheterocycles of the formula VIII are then reacted with sodium methylmercaptan or potassium methylmercaptan to give pyrimidines of the formula VII.

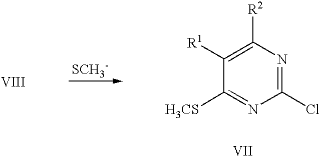

This reaction is usually carried out at temperatures of from 0° C. to 80° C. in an inert organic solvent [cf. WO 98/40379].

Suitable solvents are ethers, such as diethyl ether, diisopropyl ether, tert-butyl methyl ether, dioxane, anisole and tetrahydrofuran. Preference is given to tetrahydrofuran. It is also possible to use mixtures of the solvents mentioned.

The starting materials are generally reacted with one another in equimolar amounts.

The mercaptan can also be generated in situ by reacting the corresponding alkoxide with methylthiol in methanol at room temperature [Recl. Trav. Chim. Pays-Bas 61 (1942), 291]. Pyrimidines of the formula VII can also be prepared by the following route:

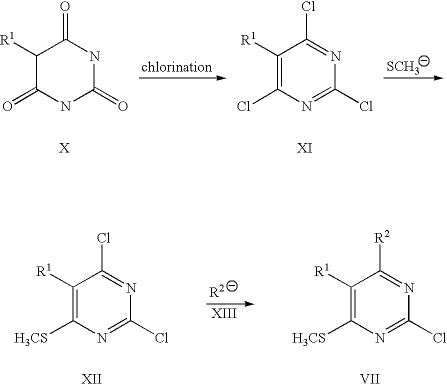

Commercially available tricarbonyl compounds of the formula X are reacted with chlorinating agents, such as, for example, phosphorus oxychloride, sulfuryl chloride or benzenephosphonic acid dichloride, in the presence of an organic base, such as, for example, triethylamine or N,N-dimethylaminopyridine, to give the corresponding trichloropyrimidine [cf. DE 196 51 310, M. M. Robinson, J. Am. Chem. Soc. 80 (1058), 5481].

The trichloro compound of the formula XI is then reacted with one equivalent of sodium mercaptan or potassium mercaptan in an inert organic solvent, such as, for example, tetrahydrofuran or dioxane, at 0° C.-80° C. to give the corresponding thioether of the formula XII. Here, it is also possible to generate the mercaptan as described above in situ.

The thioether of the formula XII is then reacted with one equivalent of the corresponding nucleophile R^{2⊖} of the formula XIII. For R²=alkoxy, alkenyloxy, alkynyloxy, haloalkoxy, alkoxyalkyl, alkylamino, dialkylamino, alkylthio, haloalkylthio, the corresponding alcohols, amines, thiols are, if appropriate in the presence of a base, such as, for example, an alkali metal carbonate or alkaline earth metal carbonate or a corresponding hydroxide, reacted in an inert organic solvent, such as, for example, tetrahydrofuran, acetonitrile or N,N-dimethylformamide, at 0° C.-130° C. to give pyrimidines of the formula VII [J. March, Advanced Organic Chemistry 1992, 641 f.].

In the case of R²=halogen or cyano, the corresponding metal salts R²-M are employed.

In the case of R² =alkyl, the corresponding organometallic compounds, such as Grignard reagents or organolithium compounds, are employed.

The pyrimidines of the formula VII obtained by these routes are reacted with azoles of the formula V to give N-heterocyclyl-substituted pyrimidines of the formula VI:

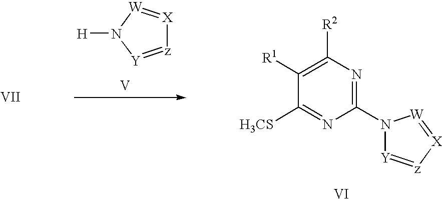

This reaction is usually carried out at temperatures of from 0° C. to 130° C. in an inert organic solvent in the presence of a base (cf. WO 98/40379].

Suitable solvents are, for example, ethers, such as diethyl ether, diisopropyl ether, tert-butyl methyl ether, dioxane, anisole and tetrahydrofuran, nitriles, such as acetonitrile and propionitrile, dimethylformamide or dimethyl sulfoxide. Preference is given to dimethylformamide.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate. Preference is given to alkali metal and alkaline earth metal carbonates, for example potassium carbonate.

The bases are generally employed in equimolar amounts or in excess.

It may furthermore be advantageous to carry out the reaction in the presence of a catalytic amount, for example 0.1 eq., of a base, such as, for example, DABCO (1,4-diazabicyclo[2.2.2]octane) (cf. J. A. Lin, E. W. McLean, J. L. Kelley, J.Chem.Soc., Chem. Comm. 1994, 8, 913-914).

The starting materials are generally reacted with one another in equimolar amounts.

The N-heterocyclyl-substituted pyrimidines of the formula VI are then reacted with an oxidizing agent to give compounds of the formula II:

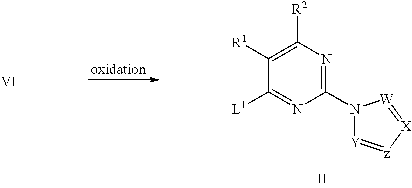

This reaction is usually -carried out at temperatures of from 0° C. to 60° C. in an inert organic solvent (cf. J. March, Advanced Organic Chemistry, 1992, 1201-1203.].

L¹ is a nucleophilically displaceable leaving group, for example alkylsulfonyl, preferably methylsulfonyl.

Suitable oxidizing agents are metachloroperbenzoic acid, hydrogen peroxide, sodium peroxide or Oxone®. Preference is given to metachloroperbenzoic acid.

It may be advantageous to carry out the reaction in the presence of a catalyst such as, for example, sodium tungstate.

Suitable solvents are halogenated hydrocarbons, such as methylene chloride, chloroform and chlorobenzene, and alcohols, such as methanol, ethanol, n-propanol, isopropanol, n-butanol and tert-butanol.

The starting materials are generally reacted with one another in equimolar amounts. It may be advantageous to employ an excess of oxidizing agent, based on VI.

The compounds of the formula II are then reacted with a thiophene derivative of the formula III to give N-heterocyclyl-substituted thienyloxypyrimidines of the formula I:

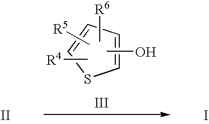

This reaction is usually carried out at temperatures of from 0° C. to 130° C., preferably from 25° C. to 40° C., in an inert organic solvent in the presence of a base [cf. WO 98/40379].

Suitable solvents are ethers, such as diethyl ether, diisopropyl ether, tert-butyl methyl ether, dioxane, anisole and tetrahydrofuran, nitriles, such as acetonitrile and propionitrile, alcohols, such as methanol, ethanol, n-propanol, isopropanol, n-butanol and tert-butanol, and also dimethyl sulfoxide and dimethylformamide, particularly preferably dimethylformamide.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate.

Particular preference is given to alkali metal and alkaline earth metal carbonates.

The bases are generally employed in equimolar amounts.

The starting materials are generally reacted with one another in equimolar amounts.

Work-up can be carried out in a manner known per se. The reaction mixture is, for example, acidified with dilute mineral acid, such as 5% strength hydrochloric acid or sulfuric acid, and extracted with an organic solvent, for example methylene chloride or ethyl acetate. The organic extract may be extracted with 5-10% strength alkali metal carbonate solution, for example with sodium carbonate or potassium carbonate solution. The aqueous phase is acidified and the precipitate that is formed is filtered off with suction and/or extracted with methylene chloride or ethyl acetate, dried and concentrated.

Process B

Dichloroheterocycles of formula VIII are reacted with thiophene derivatives of the formula III to give thienyloxypyrimidine derivatives of the formula IV:

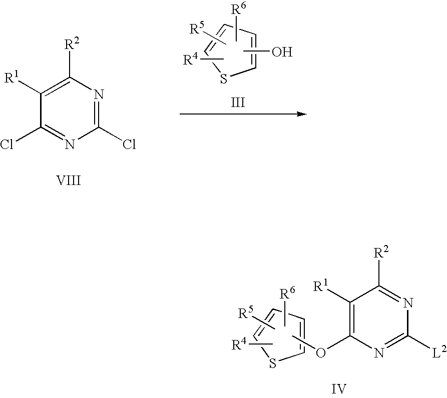

This reaction is usually carried out under the same conditions as described above for the conversion of II into I.

L² is a leaving group, such as halogen, for example chlorine, bromine or iodine, C₁-C₄-alkylsulfonyl, C₁-C₄-haloalkylsulfonyloxy or trialkylammonium; preference is given to chlorine, C₁-C₄-alkylsulfonyl, such as, for example, methylsulfonyl, or C₁-C₄-haloalkylsulfonyloxy, such as, for example, trifluoromethylsulfonyloxy.

The thienyloxypyrimidine derivatives of the formula IV are then reacted with azoles of the formula V to give N-heterocyclyl-substituted thienyloxypyrimidines of the formula I:

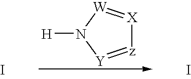

This reaction is carried out in the presence of a base, usually under the same conditions as described above for the conversion of VII in VI.

The reaction can be carried out, for example, as follows:

In the case of pyrazoles and imidazoles in the presence of a base, such as, for example, potassium carbonate in dimethylformamide;

in the case of pyrroles in the presence of a base, such as, for example, potassium tert-butoxide and DABCO in tetrahydrofuran;

in the case of triazoles in the presence of a base, such as, for example, potassium carbonate and DABCO in acetonitrile.

In addition, this reaction can also be carried out with palladium catalysis. In this case, the reaction is usually carried out at temperatures of from 25° C. to 130° C. in an inert organic solvent in the presence of a base (cf. J. F. Hartwig et al., J. Am. Chem. Soc. 120 (1998), 827-828; S. L. Buchwald et al., J. Organomet. Chem. 576 (1999), 125-146].

Suitable catalysts are, for example, palladium ligand complexes in which the palladium is present in oxidation state 0, metallic palladium, if appropriate on a support, and, preferably, palladium(II) salts. The reaction with palladium(II) salts and metallic palladium is preferably carried out in the presence of complex ligands.

Suitable palladium(0) complex ligands are, for example, tetrakis(triphenylphosphine)palladium, palladium(diphenylphosphinoferrocene)dichloride{[PdCl₂(dppf)]} or tris(dibenzylideneacetone)dipalladium(Pd₂(dba)₃.

Suitable palladium(II) salts are, for example, palladium acetate and palladium chloride. The reaction is preferably carried out in the presence of complex ligands, such as, for example, triphenylphosphine.

Complex palladium salts can be prepared in a manner known per se starting from commercially available palladium salts, such as palladium dichloride or palladium diacetate, and the corresponding phosphines, such as, for example, triphenylphosphine or 1,2-bis(diphenylphosphino)ethane. Many of the complex palladium salts are also commercially available. Preferred palladium salts are[(R)-(+)-2,2′-bis(diphenylphosphino)-1,1′-binaphthyl]palladium(II)chloride, bis(triphenylphosphine)palladium(II)acetate and, in particular, bis(triphenylphosphine)palladium(II)chloride.

The palladium catalyst is generally employed in a concentration of from 0.05 to 5 mol %, preferably 1-3 mol %.

Suitable solvents are aromatic hydrocarbons, such as toluene, o-, m- and p-xylene, ethers, such as diethyl ether, diisopropyl ether, tert-butyl methyl ether, dioxane, anisole and tetrahydrofuran, and also dimethylformamide.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, and also alkali metal and alkaline earth metal alkoxides, such as sodium methoxide, sodium ethoxide, potassium ethoxide, potassium tert-butoxide and dimethoxymagnesium.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to employ an excess of V, based on IV.

Work-up can be carried out in a manner known per se to afford the product.

Process C

It is also possible to synthesize the nitrogen heterocycle directly from a corresponding aminopyrimidine. In this case, N-heterocyclyl-substituted pyrimidines are obtained, which can then be modified further according to the reactions shown above. This variant C is, by way of example, demonstrated using an aminopyridine of the formula XIV, reaction of which gives N-heterocyclyl-substituted pyrimidines of the formula XV. However, the heterocycle can also be synthesized at a different stage of variant A or B shown above.

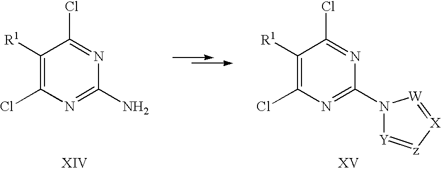

The reactions mentioned below are known from the literature and described, inter alia, in T. Eicher, S. Hauptmann, The Chemistry of Heterocycles and in J. A. Joule, K. Mills, Heterocyclic Chemistry.

Pyrrole derivatives can be prepared by reacting the corresponding primary amine with a dicarbonyl compound in a Paal-Knorr synthesis. Using β-ketoesters and primary amines, 3-alkoxycarbonyl- or 3-acyl-substituted pyrroles are obtained via α-halocarbonyl compounds (Hantzsch synthesis). Pyrimidyl-substituted pyrroles are obtained by reaction of aliphatic or aromatic amines with dimethoxytetrahydrofuran or 1,4-dichloro-1,4-dimethoxybutane.

Imidazoles are obtained by reaction of isocyanates with imines under basic conditions, or by reaction of 2-bromoketones with amidine or guanidine derivatives in the presence of a base. The corresponding amidine or guanidine derivatives are prepared similarly to processes known from the literature from XIV.

Pyrazoles can be synthesized by initially converting the primary amine into the diazonium compound. After hydrogenation, the corresponding pyrimidinehydrazine derivative is obtained which, using 1,3-dicarbonyl compounds, enole esters or 1-alkynylketones in a cyclocondensation, gives the desired pyrazole.

1,2,3-triazoles can be obtained by reacting azides with alkynes or CH-acidic compounds in the presence of an alkoxide. The condensation of azides with acyl-Wittig reagents allows a regio-specific synthesis of 1,5-disubstituted 1,2,3-triazoles.

1,2,4-triazoles are obtained by reacting pyrimidinehydrazine derivatives with diacylamines in the presence of weak acids or by reacting N,N′-diacylhydrazine with aminopyrimidine derivatives in the presence of phosphorus pentoxide.

Tetrazoles can be synthesized by [3+2] cycloaddition of azides with nitriles or an activated amide. Reaction of aryl thioisocyanates with azides or nitrosation of pyrimidylammonium hydrazones are alternative methods for synthesizing substituted tetrazoles.

Thienyloxypyrimidine derivatives of the formula IV

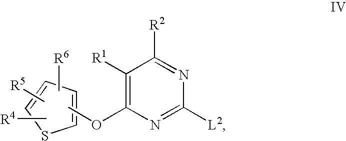

where R¹, R², R⁴, R⁵ and R⁶ are as defined in claim 1 and L² is a nucleophilically displaceable leaving group, such as halogen, for example chlorine, bromine or iodine, Cₗ-C₄-alkylsulfonyl, C₁-C₄-haloalkylsulfonyloxy or trialkylammonium, preferably chlorine, C₁-C₄-alkylsulfonyl, such as, for example, methylsulfonyl, or C₁-C₄-haloalkylsulfonyloxy, such as, for example, trifluoromethylsulfonyloxy, are key intermediates in the synthesis of triphenyloxypyrimidine derivatives of the formula I according to the invention according to process B.

With respect to the variables, the particularly preferred embodiments of the intermediates correspond to those of the radicals R¹, R², R⁴, R⁵ and R⁶ of formula I.

Particular preference is given to intermediates of the formula IV in which

R¹ is hydrogen, halogen, cyano, C₁-C₆-alkyl, C₁-C₆-haloalkyl or C₁-C₆-alkoxy;

particularly preferably hydrogen, halogen or C₁-C₆-alkyl; with particular preference hydrogen, fluorine, chlorine or methyl;

R² is hydrogen, halogen, cyano, Cₗ-C₆-alkyl, C₁-C₆-haloalkyl, C₁-C₆-alkoxy, C₁-C₆-haloalkoxy, C₁-C₆-alkoxy-C₁-C₄-alkyl, C₁-C₆-alkylthio or COOR⁷;

particularly preferably hydrogen, halogen, cyano, C₁-C₆-alkyl, C₁-C₆-haloalkyl, C₁-C₆-alkoxy, C₁-C₆-alkoxy-C₁-C₄-alkyl or C₁-C₆-alkylthio;

with particular preference hydrogen, halogen, C₁-C₆-alkyl or C₁-C₆-haloalkyl;

very preferably hydrogen, fluorine, chlorine, methyl or trifluoromethyl;

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl or C₁-C₆-haloalkylsulfonyl;

particularly preferably hydrogen, halogen, cyano, C₁-C₆-alkyl, C₁-C₆-haloalkyl, C₁-C₆-alkoxy, C₁-C₆-alkylsulfonyl or C₁-C₆-haloalkylsulfonyl;

with particular preference hydrogen, halogen, C₁-C₆-haloalkyl, C₁-C₆-alkylsulfonyl or C₁-C₆-haloalkylsulfonyl;

very preferably hydrogen, fluorine, chlorine, trifluoromethyl or methylsulfonyl.

TABLE 3

| | |
|---|---|
| | IV.1 |
| 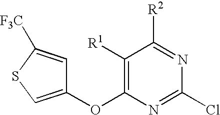 | |

| No. | R¹ | R² | ¹H-NMR/CDCl₃ or m.p. [° C.] |
|---|---|---|---|
| IV.1.1 | H | CH₃ | δ=2.53(s, 3H), 6.71(s, 1H), 7.35(1H, |
| | | | J=1.77Hz) |
| IV.1.2 | CH₃ | H | δ=2.29(s, 3H), 7.39(m, 1H), 7.45(1H, |
| | | | J=1.77Hz), 8.31(m, 1H). |
| IV.1.3 | H | H | δ=2.43(3H, s), 6.58(1H, d)7.32(1H, d), |
| | | | 7.38(1H, m), 8.4(1H, d). |
| IV.I.4 | CH₃ | CH₃ | δ=2.25(3H, s), 2.50(3H, s), 7.34(1H, s), |
| | | | 7.40(1H, s). |

TABLE 4

| | |
|---|---|
| | IV.2 |
| 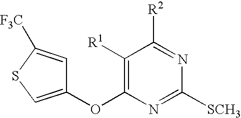 | |

| No. | R¹ | R² | ^{1H}-NMR/CDCl₃ or m.p. [° C.] |
|---|---|---|---|
| IV.2.1 | H | Cl | δ=2.43(3H, s), 6.60(1H, s), 7.30(1H, d) |
| | | | 7.35(1H, m). |
| IV.2.2 | H | OCH₃ | δ=2.43(3H, s), 3.96(3H, s), 6.86(1H, s) |
| | | | 7.24(1H, d), 7.35(1H, m). |
| IV.2.3 | H | CF₃ | δ=2.48(3H, s), 6.90(1H, s), 7.37(1H, d) |
| | | | 7.38(1H, m). |

### PREPARATION EXAMPLES

### 5-methyl-2-[3-(trifluoromethyl)-1H-pyrazol-1-yl]-4-{[5-(trifluoromethyl)-3-thienyl]oxy}pyrimidine

Process A:

### 2,4-dichloro-5-methylpyrimidine

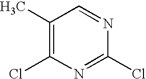

At room temperature, 2 ml of N,N-dimethylformamide were slowly added to a solution of 60 ml of phosphorus oxychloride. The mixture was stirred for 10 min, 10 g (79.3 mmol) of thymine were then added a little at a time, and the mixture was stirred for another 10 min. The mixture was then slowly heated to 105° C. and stirred at this temperature for 3 h. The mixture was hydrolyzed using 800 ml of H₂O, and the solution was extracted with ethyl acetate. The organic phases were separated off, combined, washed with H₂O and dried, and the solvent was removed. This gave 12.3 g (95% of theory) of the title compound.

1H-NMR (400 MHz, CDCl₃) δ=2.35 (s, 3H), 8.45 (s, 1H)

### 2-chloro-5-methyl-4-(methylthio)pyrimidine

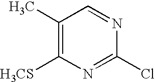

With ice-cooling, 7.3 g (98.02 mmol) of NaSCH₃ were added a little at a time to a solution of 13.77 g (84.5 mmol) of 2,4-dichloro-5-methylpyrimidine in 100 ml of tetrahydrofuran. The reaction mixture was stirred at room temperature for 6 h. The reaction mixture was then hydrolyzed with H₂O and extracted with ethyl acetate. The combined organic phases were washed with H₂O and dried, and the solvent was removed by distillation. This gave 13.32 g (90% of theory) of the title compound as a colorless solid.

m.p.: 73-76° C. 1H-NMR (400 MHz, CDCl₃) δ=2.15 (s, 3H), 2.60 (s, 3H), 8.01 (s, 1H).

### 5-methyl-4-methylthio-2-[3-(trifluoromethyl)-1H-pyrazol-1-yl]pyrimidine

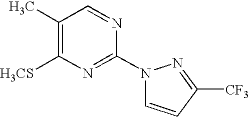

A mixture of 12.32 g (70.5 mmol) of 2-chlorine-5-methyl-4-(methylthio)pyrimidine, 11.5-g (84.6 mmol) of 3-trifluoromethyl-1H-pyrazole, 24.3 g (176.4 mmol) of sodium carbonate and 150 ml of N,N-dimethylformamide was heated at 100° C. for 6 h and then stirred at 25° C. overnight. The phases were then separated, the organic phase was washed and dried and the solvent was removed. This gave 18.15 g (94% of theory) of the title compound.

1H-NMR (400 MHz, CDCl₃) δ=2.25 (s,3H), 2.65 (s, 3H), 6.73 (s, 1H), 8.23 (s, 1H), 8.63 (s, 1H).

### 2-(4,5-dichloro-1H-imidazol-1-yl)-5-methyl-4-(methylthio)pyrimidine

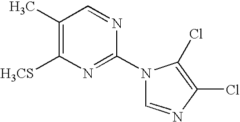

A mixture of 8.22 g (46.9 mmol) of 2-chloro-5-methyl-4-(methylthio)pyrimidine, 8.5 g (61.8 mmol) of 4,5-dichloro-1H-imidazole, 17.8 g (128.8 mmol) of sodium carbonate and 150 ml of N,N-dimethylformamide was heated at 100° C. for 12 h. The phases were then separated, the organic phase was washed and dried and the solvent was removed. This gave 18.15 g (94% of theory) of the title compound.

1H-NMR (400 MHz, CDCl₃) δ=2.25 (s, 3H), 2.62 (s, 3H), 8.15 (s, 1H), 8.35 (s, 1H).

### 5-methyl-4-methylsulfone-2-[3-(trifluoromethyl)-1H-pyrazol-1-yl]-pyrimidine

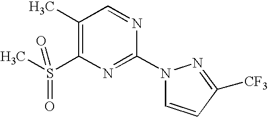

33.6 g (116.7 mmol) of 60% strength metachloroperbenzoic acid were added to a solution of 16 g (58.3-mmol) of 5-methyl-4-methylthio-2-[3-(trifluoromethyl)-1H-pyrazol-1-yl]-4-pyrimidine in 150 ml of methylene chloride. The reaction mixture was stirred at 25° C. for 4 h and then filtered. The filtrate was washed with sodium hydrogen sulfide, sodium bicarbonate and H₂O and dried. The solvent was removed by distillation. This gave 16 g (90% of theory) of the title compound as a colorless solid.

m.p.: 107-111° C. 1H-NMR (400 MHz, CDCl₃) δ=2.72 (s, 3H), 3.47 (s, 3H), 6.79 (s, 1H), 8.58 (s, 1H), 8.85 (s, 1H).

### 5-methyl-2-[3-(trifluoromethyl)-1H-pyrazol-1-yl]-4-{[5-(tri-fluoromethyl)-3-thienyl]oxy}pyrimidine

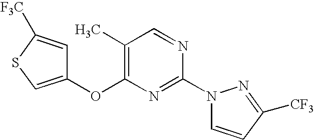

A mixture of 1 g (3.27 mmol) of 5-methyl-4-methylsulfone-2-[3-(trifluoromethyl)-1H-pyrazol-1-yl]pyrimidine, 0.71 g (4.24 mmol) of 5-(trifluoromethyl)-3-hydroxythiene and 0.9 g (6.53 mmol) of sodium carbonate in 20 ml of N,N-dimethylformamide was stirred at 25° C. for 2 h. The phases were separated, the organic phase was washed and dried and the solvent was removed. The crude product was purified chromatographically (cyclohexane/ethyl acetate 10:1). This gave 0.7 g (54% of theory) of the title compound as a colorless solid.

m.p.: 113-115° C. 1H-NMR (400 MHz, CDCl₃) δ=2.35 (s, 3H), 6.70 (s, 1H), 7.48 (s, 2H), 8.28 (s, 1H), 8.52 (s, 1H).

In addition to the above compound, Tables 5 and 6 list further N-heterocyclyl-substituted pyrimidines of the formula I which were prepared or are preparable in a similar manner.

TABLE 5

| | |
|---|---|
| | I.1 |
| 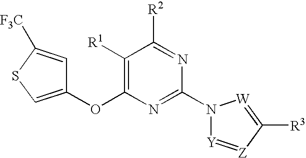 | |

| No. | R¹ | R² | R³ | W | Y | Z | m.p.[° C.] or ¹H-NMR/CDCl₃ |
|---|---|---|---|---|---|---|---|
| I.1.1 | CH₃ | H | CF₃ | N | CH | CH | 113-115 |
| I.1.2 | CH₃ | H | CF₃ | CH | CH | N | |
| I.1.3 | CH₃ | H | CF₃ | N | CH | N | |
| I.1.4 | CH₃ | H | Cl | CCl | CH | N | 113-115 |
| I.1.5 | CH₃ | H | H | CNO₂ | CCH₃ | N | 126-129 |
| I.1.6 | CH₃ | H | C(CH₃)₂ | N | CBr | N | δ=1.40(9H, s), 2.36 |
| | | | | | | | (3Hs), 7.36(1H, s), |
| | | | | | | | 7.44(1H, s), 8.64 |
| | | | | | | | (1H, s). |
| I.1.7 | CH₃ | H | Br | N | CNO₂ | N | |
| I.1.8 | H | CH₃ | CF₃ | N | CH | CH | |
| I.1.9 | H | CH₃ | CF₃ | CH | CH | N | |
| I.1.10 | H | CH₃ | CF₃ | N | CH | N | |
| I.1.11 | H | CH₃ | Cl | CCl | CH | N | 97-99 |
| I.1.12 | H | CH₃ | H | CNO₂ | CCH₃ | N | |
| I.1.13 | H | CH₃ | C(CH₃)₃ | N | CBr | N | |
| I.1.14 | H | CH₃ | Br | N | CNO₂ | N | |
| I.1.15 | H | H | CF₃ | N | CH | CH | 10-111 |
| I.1.16 | H | OCH₃ | CF₃ | N | CH | CH | 74-79 |
| I.1.17 | H | CF₃ | CF₃ | N | CH | CH | 164-169 |
| I.1.18 | CH₃ | CH₃ | CF₃ | N | CH | CH | 105-116 |
| I.1.19 | H | H | Cl | CCl | CH | N | δ=6.96(1H, d), 7.34 |
| | | | | | | | (1H, d), 7.41(1H, m), |
| | | | | | | | 8.31(1H, s), 8.63 |
| | | | | | | | (1H, d). |
| I.1.20 | CH₃ | CH₃ | Cl | CCl | CH | N | δ=2.17(3H, s), 2.63 |
| | | | | | | | (3H, s), 7.38(1H, d), |
| | | | | | | | 7.40(1H, m), 7.57 |
| | | | | | | | (1H, s). |
| I.1.21 | CH₃ | H | NO₂ | CH | CCH₃ | N | 126-129 |
| I.1.22 | CH₃ | CH₃ | NO₂ | CH | CCH₃ | N | δ=2.34(3H, s), 2.47 |
| | | | | | | | (3H, s), 2.58(3H, s) |
| | | | | | | | 7.21(1H, d), 7.33 |
| | | | | | | | (1H, m), 8.55(1H, s). |
| I.1.23 | H | CH₃ | NO₂ | CH | CCH₃ | N | δ=2.53(3H, s), 2.60 |
| | | | | | | | (3H, s), 6.81(1H, s), |
| | | | | | | | 7.23(1H, d), 7.33 |
| | | | | | | | (1H, m), 8.60(1H, s). |
| I.1.24 | H | OCH₃ | C(CH₃)₃ | N | CBr | N | 126-127 |
| I.1.25 | H | CH₃ | S-n-C₆H₁₄ | N | CH | N | δ=0.90(3H, t), 1.30-1.48 |
| | | | | | | | (6H, m), 1.56 |
| | | | | | | | (3H, s)1.76-1.81 |
| | | | | | | | (2H, m), 2.58(3H, s), |
| | | | | | | | 3.20(2H, t), 7.34 |
| | | | | | | | 1H, d), 7.42(1H, s), |
| | | | | | | | 8.94(1H, s). |
| I.1.26 | CH₃ | H | S-n-C₆H₁₄ | N | CH | N | δ=0.89(3H, t), 1.30-1.48 |
| | | | | | | | (6H, m), 1.56 |
| | | | | | | | (3H, s), 1.76-1.81 |
| | | | | | | | (2H, m), 2.63(3H, s), |
| | | | | | | | 3.19(2H, t), 7.31 |
| | | | | | | | (1H, d), 7.41(1H, m), |
| | | | | | | | 8.51(1H, s), 9.01 |
| | | | | | | | (1H, s). |
| I.1.27 | CH₃ | H | CF₃ | N | N | CH | δ=2.42(3H, d), 7.46 |
| | | | | | | | (1H, m), 7.71(1H, d), |
| | | | | | | | 8.56(1H, d), 8.64 |
| | | | | | | | (1H, d). |
| I.1.28 | H | H | CF₃ | N | N | CH | δ=7.08(1H, d), 7.44 |
| | | | | | | | (1H, m), 7.69(1H, d) |
| | | | | | | | 8.71(1H, s), 8.75 |
| | | | | | | | (1H, d). |
| I.1.29 | CH₃ | H | CF₃ | CH | N | N | δ=7.57(1H, m), 7.91 |
| | | | | | | | (1H, d), 8.15(1H, s), |
| | | | | | | | 8.59(1H, d). |
| I.1.30 | H | H | CF₃ | CH | N | N | δ=7.06(1H, d), 7.53 |
| | | | | | | | (1H, m), 7.83(1H, d), |
| | | | | | | | 8.19(1H, s), 8.79 |
| | | | | | | | (1H, d). |

TABLE 6

| | |
|---|---|
| | I.2 |
| 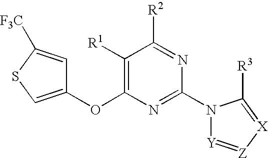 | |

| | | | | | | | m.p.[° C.] or |
|---|---|---|---|---|---|---|---|
| No. | R¹ | R² | R³ | X | Y | Z | ¹H-NMR/CDCl₃ |
| I.2.1 | H | CH₃ | CH₃ | N | CH | N | 172-178 |
| I.2.2 | CH₃ | H | CH₃ | N | CH | N | 85-87 |
| I.2.3 | CH₃ | CH₃ | CH₃ | N | CH | N | δ= |
| | | | | | | | 2.50(3H, s), 2.55 |
| | | | | | | | (3H, s), |
| | | | | | | | 2.60(3H, s), |
| | | | | | | | 7.35(1H, s), 7.42 |
| | | | | | | | (1H, s), |
| | | | | | | | 8.82(1H, s). |
| I.2.4 | CH₃ | H | Cl | N | CCl | N | δ= |
| | | | | | | | 2.40(3H, s), 7.40 |
| | | | | | | | (1H, s) |
| | | | | | | | 7.48(1H, s), |
| | | | | | | | 8.32(1H, s). |
| I.2.5 | CH₃ | H | CF₃ | N | CH | N | 73-75 |
| I.2.6 | H | CH₃ | CF₃ | N | CH | N | δ= |
| | | | | | | | 2.63(3H, s), 7.36 |
| | | | | | | | (1H, d), |
| | | | | | | | 7.44(1H, m), |
| | | | | | | | 7.57(1H, s), 9.12 |
| | | | | | | | (1H, s) |
| I.2.7 | H | H | CF₃ | H | CH | N | δ= |
| | | | | | | | 7.04(1H, d), 7.45 |
| | | | | | | | (1H, m), |
| | | | | | | | 7.50(1H, d), |
| | | | | | | | 8.74(1H, d), 9.05 |
| | | | | | | | (1H, s) |
| I.2.8 | CH₃ | H | CH₃ | CH | CCH₃ | CH | δ= |
| | | | | | | | 1.56(6H, s), 2.16 |
| | | | | | | | (3H, s), |
| | | | | | | | 7.18(1H, d) |
| | | | | | | | 7.25(1H, d), 7.30 |
| | | | | | | | (1H, d), |
| | | | | | | | 7.32(1H, m), |
| | | | | | | | 8.26(1H, s) |
| I.2.9 | H | CH₃ | CN | CH | CH | CH | 98-100 |
| I.2.10 | CH₃ | H | CN | CH | CH | CH | 130-136 |
| I.2.11 | H | H | C(═O)CF₃ | CH | CH | CH | δ= |
| | | | | | | | 6.52(1H, m), 7.05 |
| | | | | | | | (1H, d), |
| | | | | | | | 7.32(1H, s), |
| | | | | | | | 7.30(1H, s), 7.48 |
| | | | | | | | (1H, m), |
| | | | | | | | 7.62(1H, m) |
| | | | | | | | 8.67(1H, d) |
| I.2.12 | CH₃ | H | C(═O)CF₃ | CH | CH | CH | δ= |
| | | | | | | | 2.30(3H, s), 7.32 |
| | | | | | | | 1H, m), |
| | | | | | | | 7.15-7.25 |
| | | | | | | | (2H, m), |
| | | | | | | | 7.40(1H, s) |
| | | | | | | | 7.45(1H, s)8.34 |
| | | | | | | | (1H, s). |

Biological Activity

The N-heterocyclyl-substituted thienyloxypyrimidines of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and harmful grasses in crops such as wheat, rice, maize, soya and cotton without causing any significant damage to the crop plants. This effect is mainly observed at low rates of application.

Depending on the application method used, the compounds of the formula I or the herbicidal compositions comprising them can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

The compounds of the formula I, or the herbicidal compositions comprising them, can be used for example in the form of ready-to-spray aqueous solutions, powders, suspensions, also highly-concentrated aqueous, oily or other suspensions or dispersions, emulsions, oil dispersions, pastes, dusts, materials for broadcasting or granules, by means of spraying, atomizing, dusting, broadcasting or watering. The use forms depend on the intended aims; in any case, they should ensure a very fine distribution of the active compounds according to the invention.

The herbicidal compositions comprise a herbicidally effective amount of at least one compound of the formula I or an agriculturally useful salt of I and auxiliaries customary for formulating crop protection agents.

Essentially, suitable inert auxiliaries include: mineral oil fractions of medium to high boiling point, such as kerosene and diesel oil, furthermore coal tar oils and oils of vegetable or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates consisting of active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl polyglycol ether or tributylphenyl polyglycol ether, alkylaryl polyether alcohols, isotridecyl alcohol, fatty alcohol ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise from about 0.001 to 98% by weight, preferably 0.01 to 95% by weight of at least one active compound. The active compounds are employed in a purity of from 90% to 100%, preferably from 95% to 100% (according to the NMR spectrum).

The production of such preparations is illustrated by the following formulation examples:

I. 20 parts by weight of an active compound of the formula I are dissolved in a mixture consisting of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II. 20 parts by weight of an active compound of the formula I are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III. 20 parts by weight of an active compound of the formula I are dissolved in a mixture consisting of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

IV. 20 parts by weight of an active compound of the formula I are mixed thoroughly with 3 parts by weight of sodium diisobutylnaphthalenesulfonate, 17 parts by weight-of the sodium salt of lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V. 3 parts by weight of an active compound of the formula I are mixed with 97 parts by weight of finely divided kaolin. This gives a dust which comprises 3% by weight of the active compound.

VI. 20 parts by weight of an active compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII. 1 part by weight of an active compound of the formula I is dissolved in a mixture consisting of 70 parts by weight of cyclohexanone, 20 parts by weight of ethoxylated isooctylphenol and 10 parts by weight of ethoxylated castor oil. This gives a stable emulsion concentrate.

VIII. 1 part by weight of an active compound of the formula I is dissolved in a mixture of 80 parts by weight of cyclohexanone and 20 parts by weight of Wettol^{R} EM 31 (=nonionic emulsifier based on ethoxylated castor oil). This gives a stable emulsion concentrate.

The compounds of the formula I or the herbicidal compositions can be applied pre- or post-emergence. If the active compounds are less well tolerated by certain crop plants, application techniques may be used in which the herbicidal compositions are sprayed, with the aid of the spraying equipment, in such a way that they come into contact as little as possible, if at all, with the leaves of the sensitive crop plants, while the active compounds reach the leaves of undesirable plants growing underneath, or the bare soil surface (post-directed, lay-by).

The application rates of the compound of the formula I are from 0.001 to 3.0, preferably from 0.01 to 1.0 kg/ha of active substance (a.s.), depending on the control target, the season, the target plants and the growth stage.

To widen the activity spectrum and to achieve synergistic effects, the N-heterocyclyl-substituted thienyloxypyrimidines of the formula I may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (hetero)aryloxyalkanoic acids and their derivatives, benzoic acid and its derivatives, benzothiadiazinones, 2-(hetaroyl/aroyl)-1,3-cyclohexanediones, heteroarylaryl ketones, benzylisoxazolidinones, meta-CF3-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ether, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds of the formula I, alone or else concomitantly in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

Use Examples

The herbicidal activity of the N-heterocyclyl-substituted thienyloxypyrimidines of the formula I was demonstrated by the following greenhouse experiments:

The cultivation containers used were plastic flower pots containing loamy sand with approximately 3.0% of humus as the substrate. The seeds of the test plants were sown separately for each species.

For the pre-emergence treatment, directly after sowing the active compounds, which had been suspended or emulsified in water, were applied by means of finely distributing nozzles. The containers were irrigated gently to promote germination and growth and subsequently covered with transparent plastic hoods until the plants had rooted. This cover caused uniform germination of the test plants, unless this was adversely affected by the active compounds.

For the post-emergence treatment, the test plants were first grown to a height of from 3 to 15 cm, depending on the plant habit, and only then treated with the active compounds which had been suspended or emulsified in water. The test plants were for this purpose either sown directly and grown in the same containers., or they were first grown separately as seedlings and transplanted into the test containers a few days prior to treatment. The application rate for the post-emergence treatment was 0.25 or 0.125 kg of a.s. (active substance)/ha.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the above-ground parts and 0 means no damage, or normal course of growth.

The plants used in the greenhouse experiments were of the following species:

| Scientific name | Common name |
|---|---|
| Amaranthus retroflexus | pig weed |
| Chenopodium album | lamb's-quaters |
| Galium aparine | catchweed |
| Pharbitis purpurea | tall morningglory |
| Polygonum persicaria | lady's-thumb |

At application rates of 0.25 or 0.125 kg/ha, the compound No. I 1.1 (Table 5) showed very good post-emergence action against the undesirable plants Amaranthus retroflexus, Chenopodium album, Galium aparine, Pharbitis purpurea and Polygonum persicaria.

1. A N-heterocyclyl-substituted thienyloxypyrimidine of the formula I 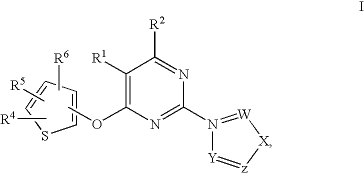
where:
W, X, Y, Z independently of one another are N or CR³, where at least one of the variables is CR³;
R¹ is hydrogen, halogen, cyano, C₁-C₆-alkyl, C₁-C₆-haloalkyl, C₁-C₆-alkoxy or C₁-C₆-haloalkoxy;
R² is hydrogen, halogen, cyano, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₃-C₆-alkenyloxy, C₃-C₆-alkynyloxy, C₁-C₆-haloalkoxy, C₁-C₆-alkoxy-C₁-C₄-alkyl, C₁-C₆-alkylamino, di-(C₁-C₄-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, COOR⁷ or CONR⁸R⁹;
R³ is hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or COOR⁷;
R⁴, R⁵, R⁶ are hydrogen, halogen, cyano, C₁-C₆-alkyl, C₁-C₆-haloalkyl, C_{-C}₆-alkoxy, C₁-C₆-haloalkoxy, C₁-C₆-alkylthio, C₁-C₆-haloalkylthio, C₁-C₆-alkylsulfonyl or C₁-C₆-haloalkylsulfonyl;
R⁷ is hydrogen, C₁-C₄-alkyl, C₃-C₄-alkenyl, C₃-C₄-alkynyl or C₁-C₄-haloalkyl;
R⁸ is hydrogen, C₁-C₄-alkyl, C₃-C₄-alkenyl, C₃-C₄-alkynyl or C₁-C₄-alkoxy;
R⁹ is hydrogen, C₁-C₄-alkyl, C₃-C₄-alkenyl or C₃-C₄-alkynyl;
and its agriculturally useful salts.

2. A N-heterocyclyl-substituted thienyloxypyrimidine of the formula I as claimed in claim 1 where
W, X, Y, Z independently of one another are N or CR³, where at most one of the variables is N.

3. A N-heterocyclyl-substituted thienyloxypyrimidine of the formula I as claimed in claim 1 where
R¹ is hydrogen, halogen, cyano, C₁-C₆-alkyl, C₁-C₆-haloalkyl or C₁-C₆-alkoxy;
R² is hydrogen, halogen, cyano, C₁-C₆-alkyl, C₁-C₆-haloalkyl, C₁-C₆-alkoxy, C₁-C₆-haloalkoxy, C₁-C₆-alkylthio or C₁-C₆-haloalkylthio.

4. A N-heterocyclyl-substituted thienyloxypyrimidine of the formula I as claimed in claim 1 where
R³ is hydrogen, halogen, cyano, C₁-C₆-alkyl, C₁-C₆-haloalkyl or C₁-C₆-alkoxy.

5. A N-heterocyclyl-substituted thienyloxypyrimidine of the formula I as claimed in claim 1 where
R⁴, R⁵ R⁶ are hydrogen, halogen, cyano, C₁-C₆-alkyl, C₁-C₆-haloalkyl or C₁-C₆-haloalkoxy.

6. A process for preparing N-heterocyclyl-substituted thienyloxypyrimidines of the formula I as claimed in claim 1, which comprises reacting pyrimidines of the formula II 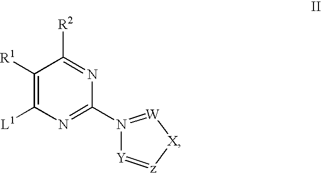
where W, X, Y, Z, R¹ and R² are as defined in claim 1 and L¹ is a nucleophilically displaceable leaving group with a thiophene derivative of the formula Ill 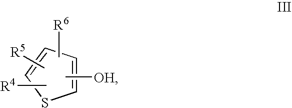
where R⁴, R⁵ and R⁶ are as defined in claim 1.

7. A process for preparing N-heterocyclyl-substituted thienyloxypyrimidines of the formula I as claimed in claim 1, which comprises reacting thienyloxypyrimidine derivatives of the formula IV 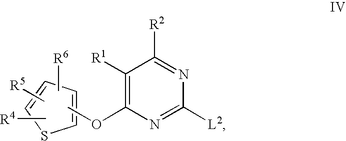
where R¹, R², R⁴, R⁵ and R⁶ are as defined in claim 1 and L² is a nucleophilically displaceable leaving group with a nitrogen heterocycle of the formula V 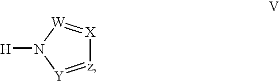
where W, X, Y and Z are as defined in claim 1.

8. A thienyloxypyrimidine derivative of the formula IV 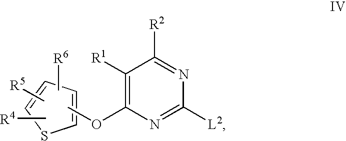
where R¹, R², R⁴, R⁵ and R⁶ are as defined in claim 1 and L² is a nucleophilically displaceable leaving group.

9. A composition, comprising a herbicidally effective amount of at least one N-heterocyclyl-substituted thienyloxypyrimidine of the formula I or an agriculturally useful salt of I as claimed in claim 1 and auxiliaries customary for formulating crop protection agents.

10. A process for preparing compositions as claimed in claim 9, which comprises mixing a herbicidally effective amount of at least one N-heterocyclyl-substituted thienyloxypyrimidine derivative of the formula I as or an agriculturally useful salt of I and auxiliaries customary for formulating crop protection agents.

11. A method for controlling undesirable vegetation, which comprises allowing a herbicidally effective amount of at least one N-heterocyclyl-substituted thienyloxypyrimidine derivative of the formula I as claimed in claim 1 or an agriculturally useful salt of I to act on plants, their habitat and/or on seeds.

12. (canceled)
